(19) United States
(12) Patent Application Publication
(10) Pub. No.: US 2024/0368284 A1
(43) Pub. Date: Nov. 7, 2024

(54) A METHOD OF IMPROVING STABILITY OF IMMUNE CHECK POINT INHIBITORS

(71) Applicant: DR. REDDY’S LABORATORIES LIMITED, Hyderabad (IN)

(72) Inventors: Murali JAYARAMAN, Kancheepuram (Dist) (IN); Saisharan K GOUD, Kamareddy (Distt) (IN); Sunil ASHOK NANKAR, Jalgaon (Distt) (IN); Maya NANATH, Palakkad (IN); Indra Kumar SIGIREDDI, Kharagpur (IN); Lovisha AGGARWAL, New Delhi (IN); Sireesha Goswamy KALIGATLA, Visakhapatnam (IN); Ravi Kumar MARIKANTY, Hyderabad (IN); Abirami S, Namakkal (IN); Giridhar SIVALANKA, Eluru (IN); Ravi Kiranmai PENMETSA, Hyderabad (IN); Suman LABALA, Srikakulam (IN); Mahesh INGALE, Solapur (IN); Puja SARKAR, Chennai (IN); Mayur Vijay DESAI, Kolhapur (IN); Prathibha Chandrashekhar KIRAVE, Pune (IN); Chetan Govindrao SHINDE, Ahmednagar (IN)

(21) Appl. No.: 18/688,663

(22) PCT Filed: Sep. 2, 2022
(86) PCT No.: PCT/IN2022/050787

§ 371 Date: Mar. 1, 2024

(30) Foreign Application Priority Data
Sep. 3, 2021 (IN) 202141040075
Mar. 31, 2022 (IN) 202241019550

(51) Int. Cl. C07K 16/28; A61K 9/08; A61K 39/00; A61K 47/12; A61K 47/18; A61K 47/26

(52) U.S. Cl. CPC C07K 16/2818; A61K 9/08; A61K 47/12; A61K 47/183; A61K 47/26; A61K 2039/505; C07K 2317/21; C07K 2317/76; C07K 2317/94

(57) ABSTRACT

The present invention relates to pharmaceutical formulations of antibodies and antigen-binding fragments against human programmed death receptor-1 (PD-1)/programmed death receptor Ligand 1 (PD-L1), and method for preparing the same. The disclosed formulations stabilizes anti-PD1/anti-PD L1 antibody from lower to higher concentrations rendering it suitable for different modes of administration (subcutaneous/intravenous).

### FIELD OF THE INVENTION

The present invention relates to stable formulations of antibodies and antigen-binding fragments against human programmed death receptor-1 (PD-1)/programmed death receptor Ligand 1 (PD-L1), and method for preparing the same.

### BACKGROUND

Over the past two decades, recombinant DNA technology has led to the commercialization of many proteins, particularly antibody therapeutics. The effectiveness of these therapeutic antibodies is majorly dependent on the stability, route of administration and their dosage forms and concentrations. This in turn, necessitates therapeutic antibodies to be formulated appropriately to retain the stability and activity of a therapeutic antibody.

Formulations for each route of administration and dosage forms may be unique and, therefore, have specific requirements. Solid dosage forms, such as lyophilized powders, are generally more stable than liquid (aqueous) formulations. However, reconstitution of the lyophilized formulation requires a significant vial overfill, care in handling and involves high production cost relative to a liquid formulation. While liquid formulations are advantageous in these and are usually preferred for injectable protein therapeutics (in terms of convenience for the end user and ease of preparation for the manufacturer), this form may not always be feasible given the susceptibility of proteins to denaturation, aggregation and oxidation under stresses such as temperature, pH changes, agitation etc., . All of these stress factors could result in the loss of biological activity of a therapeutic protein/antibody.

Antibodies which binds to the human programmed death-1 protein (PD-1) or human programmed death ligand-1 protein (PDL-1) are one of the examples of therapeutic antibodies and gained lot of importance due to it's broad spectrum in treating various oncological disorders. Most of the PD1 antibodies are IgG4 isotype antibodies and PD-L1 antibodies are IgG1 isotype antibodies. Each isotype of antibody has it's own challenges in terms of formulating to be a stable formulation. Apart from fragmentation, it is known in the art that IgG4 isotype antibodies are prone for aggregation or particle formation as compared to other IgG isotypes, especially at lower pH conditions. The particles in IgG4 isotype antibodies can be visible or sub visible based on their sizes and these can form during storage, transportation and manufacture of antibodies such as during preparation, compounding, filling, handling, inspection, or other stages of manufacturing. These particles are largely proteinaceous contaminants arising from any of the above. It is widely known and accepted fact that, particles exert a substantial impact on immunogenicity (line numbers 28-29 of 3432 Ishii-Watabe et al./Journal of Pharmaceutical Sciences 106 (2017) 3431-3437). Further, particles interfere in the bioavailability and absorption of the therapeutic antibody and thus can impact the therapeutic effectiveness of the drug.

Hence, for the reasons clear enough, approving authorities have mandated strict regulatory demands with respect to sub-visible and visible particle limits in a therapeutic antibody composition. Current US Pharmacopoeia (USP) specifications include numerical limits for visible and sub-visible particles (≥10 μm and ≥25 μm in size), and in addition, recommends determination of particle concentrations/count for the particulates of >2 μm-5 μm size ranges.

It is thus not necessary, but a mandatory requirement of the regulatory guidelines to identify the particles and characterize the visible/sub-visible particles in any therapeutic antibody composition, including the IgG4s. The objective of the invention is to address this problem of particulates, both visible and sub-visible particles and especially the latter, that prominently occur in IgG4 antibody (eg., nivolumab) during storage of aqueous formulation.

Further, it is necessary to formulate the anti-PD1/IgG4 antibodies, irrespective of the isotype variation, in appropriate buffer and/or excipients composition that stabilizes the antibodies. Additionally, factors such as visual appearance and viscosity of the antibody formulation had to be taken care while preparing any therapeutic antibody formulation. Given such complexities, there remains a continuous and constant requirement for improved alternate formulations in the domain of pharmaceutical formulations.

### SUMMARY

The present invention discloses a pharmaceutical formulation of an anti-PD-1/PD-L1 antibody. In particular, the anti-PD1 antibody is nivolumab or pembrolizumab.

The pharmaceutical formulation of the present invention discloses an anti-PD1/PD-L1 antibody or an antigen-binding fragment thereof, wherein the formulation comprises anti-PD1/PD-L1 antibody, a buffer having pH of 4.5 to 6.5 and optionally, one or more pharmaceutically acceptable excipients/stabilizers. The buffer as disclosed in the anti-PD1/PD-L1 antibody formulation is succinate buffer, or acetate buffer, citrate buffer or histidine buffer or it's derivatives or salts or combinations thereof.

In particular, the disclosed formulations of the invention stabilizes anti-PD1/PD-L1 antibody from lower to higher concentration, from about 10 mg/ml to about 200 mg/ml, rendering it suitable for different routes of administration.

In one aspect, the invention discloses a method of controlling particle formation and/or formation of charge variants and/or aggregation and/or fragmentation or deamidation of an anti-PD1/PD-L1 antibody in an anti-PD-1/PD-L1 antibody composition wherein the method comprises addition of succinate buffer or citrate buffer or acetate buffer or histidine buffer, or it's derivatives or salts or combinations thereof, to the antibody composition. The said buffer composition can be added during pre-formulation and/or at the formulation stage of the antibody production.

In addition, the invention discloses a method of controlling opalescence of an anti-PD1/anti-PDL1 antibody composition in its composition, wherein the method comprises addition of succinate buffer or citrate buffer or acetate buffer or histidine buffer or it's derivatives or salts or combinations thereof, to the antibody composition. The said buffer composition can be added during pre-formulation and/or at formulation stage of the antibody production to maintain the antibody in soluble form in the composition, thereby maintaining opalescence. Further, the opalescence of the formulations obtained from the said process matches with reference opalescence standard (ROS) II or II-III.

The invention also discloses a method to impart colloidal stability to an anti-PD1/anti-PDL1 antibody wherein the method comprises formulating the anti-PD1/PD-L1 antibody in a buffer composition comprising succinate buffer or citrate buffer or acetate buffer or histidine buffer or it's derivative or salts or combinations thereof.

The disclosed formulations of the invention exhibit stability under at least one of the following accelerated conditions that includes a temperature ranging from 25° C. to 40° C. and for a period of time ranging from 1 day to 28 days/4 weeks. The antibody in the said formulation is stable and maintains 98% or more (≥98%) of monomeric content of the antibody in the formulation even after storage for two weeks at 40° C.

In another aspect, the invention discloses a method of controlling formation of visible and sub-visible particles in an IgG4 antibody composition, the method comprises preparing the antibody composition in succinate or histidine citrate buffer or acetate buffer composition having a pH of 4.5 to 6.5 and comprising sugar, a chelating agent or an anti-oxidant, and surfactant. Specifically, the disclosed method controls formation of visible and sub visible particles even after being subjected to accelerated temperatures and various stress conditions. In particular, the disclosed method controls sub-visible particles well below the acceptable regulatory limits.

The invention further discloses a method of controlling oxidation in an IgG4 antibody composition, wherein the method comprises preparing the antibody composition in succinate or histidine-citrate buffer or acetate having pH of 4.5 to 6.5 composition further comprising a sugar, a chelating agent, an antioxidant and surfactant. In particular, the said method protects oxidation of methionine residues, Met34 and Met83 of heavy chain of nivolumab and at Met105 of CDR3 of heavy chain of pembrolizumab.

The disclosed formulations of the invention exhibits stability under one or more following stress conditions such as thermal stress, agitation, freeze-thaw, chemical induced oxidation and metal induced oxidation stress.

### DETAILED DESCRIPTION OF THE INVENTION

### Definitions

The term “about” used herein would mean and include a variation of upto 20% from the particular value.

The term “antibody” as used herein encompasses whole antibodies or any antigen binding fragment (i.e., “antigen-binding portion”) or fusion protein thereof.

The term “buffer” used herein refers to an agent which resists to any change in pH of a solution, near a chosen value, up on addition of acid or base. The buffer herein includes buffering agents, or its' derivative, or salts and/or combinations thereof.

The term “stable” formulation refers to the formulation wherein the antibody therein retains its physical stability and/or chemical stability and/or biological activity.

Stability studies provides evidence of the quality of an antibody under the influence of various environmental factors during the course of time. ICH's “Q1A: Stability Testing of New Drug Substances and Products,” states that data from accelerated stability studies can be used to evaluate the effect of short-term excursions higher or lower than label storage conditions that may occur during the shipping of the antibodies.

Various analytical methods are available for measuring the physical and chemical degradation of the antibody in the pharmaceutical formulations. An antibody “retains its physical stability” in a pharmaceutical formulation if it shows substantially no signs or minimal aggregation, precipitation and/or denaturation upon visual examination of color and/or clarity, or as measured by UV light scattering or by size exclusion chromatography. An antibody is said to “retain its chemical stability” in a pharmaceutical formulation when it shows no or minimal formation of product variants which may include variants as a result of chemical modification of antibody of interest such as deamination, oxidation etc. Analytical methods such as ion exchange chromatography and hydrophobic ion chromatography may be used to investigate the chemical product variants.

The term ‘monomer’ as used herein describes antibodies consisting of two light chains and two heavy chains. The monomer content of an antibody composition is typically analyzed by size exclusion chromatography (SEC). As per the separation principle of SEC the large molecules or molecules with high molecular weight (HMW) elute first followed by smaller or lower weight molecules. In a typical SEC profile for an antibody composition, aggregates that may include dimers, multimers, etc., elute first, followed by monomer, and the clipped antibody variants or degradants may be eluted last. In some circumstances the aggregate peak or the degradant peaks may not elute as a baseline separated peaks but instead as a shoulder or abnormal broad peaks. In order to maintain the appropriate activity of an antibody, in particular of a therapeutic antibody, it is desirable to reduce the formation of aggregate or fragmentation of products and hence control the monomer content to a target value. Ability to inhibit the formation of aggregate and degradant content as measured at various time points during stability studies may indicate the suitability of the candidate formulation for antibody of interest. TSK-GEL G3000SWXL (7.8 mm×30 cm) column from TOSCH can be used on water HPLC to perform SEC.

The term ‘main peak’ as used herein refers to the peak that elutes in abundance (major peak) during a cation exchange chromatography. The peak that elutes earlier than the main peak, during a cation exchange chromatography, with a charge that is acidic relative to the main peak is termed acidic variant peak. The peak that elutes later than the main peak, during a cation exchange chromatography, with a charge that is relatively basic than the main peak is termed as basic variant peak. The main peak content can be determined by Ion exchange chromatography (IEC). There are two modes of IEC available viz., cation and anion exchange chromatography. Negatively charged molecules bind to anion exchange resins while positively charged molecules bind to cation exchange resins. In a typical cation exchange chromatographic profile of an antibody composition acidic variants elute first followed by the main peak and thereafter lastly the basic variants will be eluted. The acidic variants are a result of antibody modifications such as deamidation of asparagine residues. The basic variants are a result of incomplete removal of C-terminal lysine residue(s). In general, in an antibody a lysine residue is present at the C-terminal end of both heavy and light chain. An antibody molecule containing lysine at both heavy and light chain is referred to as K2 variant, the antibody molecule containing lysine residue at either one of heavy and light chain is referred to as K1 variant and antibody molecule having none is K0 molecule. Carboxypeptidase B (CP-B enzyme) enzyme acts on the C-terminal lysine residues present on K2 and K1 variants and thus converting them as K0 molecules. As per circumstances of the case, the IEC analysis can be carried out for samples digested with carboxypeptidase B (CP-B) enzyme. In a typical stability study it is expected that a stable formulation leads to reduction in formation of charge variants (acidic and basic variants), during the study, and hence minimize any reduction in main peak content.

Pharmaceutically acceptable excipients/stabilizers refer to the additives or carriers, which contributes to stability of the antibody in formulation. The excipients may encompass stabilizers and tonicity modifiers. Examples of stabilizers and tonicity modifiers include, but not limited to, sugars, amino acids, salts, surfactants, polymers, or it's derivatives and/or it's combination thereof.

The term sugar/s as used herein includes sugars and sugar alcohols/polyols. Sugars can be referred to monosaccharides, disaccharides, and polysaccharides. Examples of sugars include, but are not limited to, sucrose, trehalose, glucose, dextrose, raffinose and others. Examples of sugar alcohols or polyols include, but are not limited to, mannitol, sorbitol, and others.

Surfactant refers to pharmaceutically acceptable excipients used to protect the protein formulations against various stress conditions, like agitation, shearing, exposure to high temperature etc. The suitable surfactants include but are not limited to polyoxyethylensorbitan fatty acid esters such as Tween 20™ or Tween 80™, polyoxyethylene-polyoxypropylene copolymer (e.g. Poloxamer, Pluronic), sodium dodecyl sulphate (SDS) and the like or combination thereof.

Examples of salts include, but not limited to, sodium chloride, potassium chloride, magnesium chloride, sodium thiocyanate, ammonium thiocyanate, ammonium sulfate, ammonium chloride, calcium chloride, zinc chloride and/or sodium acetate.

The term “opalescence” or “opalescent appearance” refers to the degree of turbidity detected in a solution, e.g., a protein preparation, as a function of the concentration of one or more of the components in the solution, e.g., protein and/or salt concentration. The degree of turbidity can be calculated by reference to a standard curve generated using suspensions of known turbidity. Reference standards for determining the degree of turbidity for pharmaceutical compositions can be based on the United States Pharmacopeia or European Pharmacopeia criteria. Here, in this invention to measure opalescence, first Formazine solution has been prepared by mixing equal volumes of a hydrazine sulfate solution and hexamethylenetetramine solution and then diluted to prepare various reference opalescence standards. The opalescence standards includes ROS-I, ROS-II, ROS-III and ROS-IV.

Nephelometry is a turbidometric method used to detect the presence of soluble aggregates or to indicate opalescence. The output is listed in terms of nephelometric turbidity units (NTUs).

“Pre-formulation steps” refers to any or multiple steps performed before formulating the protein into a therapeutic product. Examples of such steps include, chromatography, filtration, (ultrafiltration, sterile filtration, nano filtration, diafiltration, tangential flow filtration, depth filtration), or any other steps performed to concentrate the protein or to exchange the buffer to a different/suitable buffer. The filtration steps mentioned herein may be performed in a tangential flow filtration mode.

“Formulation steps” refers to steps which are followed after the downstream chromatographic and filtration steps to prepare a drug product from drug substance, the latter obtained from the pre-formulation steps.

The term “chelators/chelating agents” refers to a compound which can form at least one bond with a metal atom. A chelating agent is typically a multidentate ligand that can be used in compositions as a stabilizer to complex with species, which might otherwise promote instability. Exemplary agents chelating include aminopolycarboxylic acids, hydroxyaminocarboxylic acids, N-substituted glycines, 2-(2-amino-2-oxocthyl)aminoethane sulfonic acid (BES), deferoxamine (DEF), niacinamide, desoxycholates, ethylenediaminetetraacetic acid (EDTA), diethylenetriaminepentaacetic acid (DTPA), nitrilotriacetic acid (NTA), N-2-acetamido-2-iminodiacetic acid (ADA), bis(aminoethyl)glycolether, N,N,N′,N′-tetraacetic acid (EGTA), trans-diaminocyclohexane tetraacetic acid (DCTA), N-hydroxyethyliminodiacetic acid (HIMDA), N,N-bis-hydroxyethylglycine(bicine), N-(trishydroxymethylmethyl)glycine(tricine), glycylglycine, sodium desoxycholate, ethylenediamine; propylenediamine; diethylenetriamine; triethylenetetraamine (trien), ethylenediaminetetraaceto EDTA; disodium EDTA, EDTA, calcium EDTA oxalic acid and malate.

The term “antioxidant” mentioned herein refers to an agent that inhibits the oxidation of other molecules and is not part of buffer component. Examples of antioxidants herein include citrate, methionine, lipoic acid, uric acid, glutathione, tocopherol, carotene, lycopene, cysteine, phosphonate compounds, e.g., etidronic acid, desferoxamine and malate.

The term “visible particles” mentioned herein refers to insoluble particulates in a liquid composition, of size measuring greater than or equal to 100 μm (≥100 μm). Formation of these insoluble particulates formation may be caused by degradation of excipients present in the formulation and/or due to protein aggregation or degradation or from any leachates from the container holding the composition. Visible particles are typically measured by visual inspection against proper lighting by an analyst.

The term “sub-visible particles” mentioned herein refers to insoluble particulates in a liquid composition, of size measuring less than (≤100 μm), specifically the sizes ranging from 1 μm to less than 100 μm. United States Pharmacopeia, USP 788 particularly provides limitations/allowable particle count for sub visible particles sizes.

In the present invention, the sub-visible particles are measured by Micro Flow Imaging technique. Micro Flow Imaging (MFI) is an integration of microscopy, fluidics, and imaging techniques to quantify sub-visible particles and characterization of the same. Bright field images (dark image against bright background as result of reflection of the particle in the sample) are captured in successive frames as sample streams through flow cell of depth 100 μm centered in the field of view of camera of fixed magnification 5× being continuously illuminated by LED of wavelength 470 nm. The detection can be limited by particle contrast and pixels available. The measurement outcome for MFI is particle concentration (counts/mL) and shape/morphology.

### DETAILED DESCRIPTION OF THE EMBODIMENTS

The present invention discloses pharmaceutical formulations of an anti-PD1/anti-PDL1 antibody. In particular, the present invention discloses pharmaceutical formulations of IgG4 anti-PD1 antibodies in specific buffer compositions. In another aspect, the invention also provides a method to control particle formation (visible and sub-visible particles) in IgG4 anti-PD1 antibody formulation. IgG4 antibodies (eg., nivolumab, pembrolizumab), are prone to form particulate matter when being formulated as an aqueous composition. Inventors of the present invention surprisingly found that, in an IgG4 antibody, nivolumab, the particulate content in it's aqueous formulation in different buffer compositions are similar when measured by Size Exclusion Chromatography (SEC), however formation of particulates and the rate at which these particle count increase, differed between varying buffer compositions. This poses a unique problem on finalizing a stable formulation for the antibody based only on SEC measurement of aggregate content since the underlying particulate content in the composition may vary (and goes undetected by SEC) resulting in increased number of particles in the final formulation or during storage, posing a hidden risk in the therapeutic composition. The present invention identified such risk, sorted and enumerated the visible and sub-visible particulate matter in the composition to present an optimal composition/formulation with particle counts well below the statutory limits. In addition, the inventors also found that, methionine residues present at 34^{th} position and 83^{rd} position (as per Kabat numbering system) of heavy chain of nivolumab antibody is more prone for oxidation as compared to other methionine residues present in nivolumab. Similarly, another anti-PD1 antibody i.e., pembrolizumab is also prone for oxidation especially methionine residue present at 105^{th} position in CDR3 of heavy chain of the antibody. The formulation composition of the present invention is also prepared in such a way to control methionine induced oxidation in the therapeutic composition.

In another embodiment, the invention discloses a liquid pharmaceutical formulation of an anti-PD1/anti-PDL1 antibody comprising:
(i) an anti-PD-1/anti-PDL1 antibody,
(ii) a buffer having pH of about 4.5 to about 6.5
(iii) one or more stabilizers and;
(iv) a surfactant.

In the above said embodiment, the buffer is an organic buffer and/or its salts or combinations thereof.

In the above mentioned embodiment of the invention, the said organic buffer is a succinate buffer or an acetate buffer or a citrate buffer or a histidine buffer.

In an embodiment, the invention discloses a method of imparting colloidal stability to an anti-PD1/PD L1 antibody, in an anti-PD1/PDL1 antibody composition, wherein the method involves addition of succinate buffer or citrate buffer or acetate buffer or histidine buffer or it's derivatives or salts or combinations thereof, to the antibody composition during pre-formulation and/or formulation stage of the antibody production.

In yet another embodiment, the invention discloses a method of controlling formation of charge variants in an anti-PD1/PD-L1 antibody composition wherein the method comprises addition of succinate or acetate or citrate buffer or it's derivatives or salts or combination thereof to the antibody composition during pre-formulation and/or formulation stage of the antibody production.

In an embodiment, the invention discloses a method of controlling aggregation and/or fragmentation of an anti-PD1/PD-L1 antibody composition wherein the method comprises addition of succinate or acetate or citrate buffer or it's derivatives or salts or combination thereof to the antibody composition during pre-formulation and/or formulation stage of the antibody production.

In another embodiment, the invention discloses a method of controlling particle formation in an anti-PD-1/PD-L1 antibody composition, wherein the method comprises addition of succinate or acetate or citrate buffer or it's derivatives or salts or combination thereof, to the antibody composition during pre-formulation and/or formulation stage of the antibody production.

In another embodiment, the invention discloses a liquid pharmaceutical formulation of an anti-PD1 antibody/anti-PD L1 antibody comprising:
i. an anti-PD1/anti-PD L1 antibody,
ii. 10-50 mM succinate buffer or acetate buffer or citrate buffer,
iii. mannitol or trehalose or sucrose or sorbitol or sodium chloride,
iv. a chelator and
v. a surfactant.

In another embodiment, the invention discloses a liquid pharmaceutical formulation of an anti-PD1 antibody/anti-PD L1 antibody comprising:
i. an anti-PD1/anti-PD L1 antibody,
ii. 10-50 mM succinate buffer or acetate buffer or citrate buffer,
iii. mannitol or trehalose or sucrose or sorbitol or sodium chloride,
iv. an amino acid or an anti-oxidant,
v. a chelator and
vi. a surfactant.

In any of the above mentioned embodiments, the buffer includes derivatives or salts or combinations thereof, viz., the succinate buffer is a succinate buffer or a succinate-arginine buffer or a succinate-phosphate buffer and; the citrate buffer is a citrate buffer or a citrate-histidine buffer or a citrate-arginine buffer or a citrate-phosphate buffer and; the acetate buffer is an acetate buffer or an acetate-arginine buffer or an acetate-phosphate buffer.

In any of the above mentioned embodiment, the chelator is ethylenediamine tetraacetic acid (EDTA) or ethylene glycol-bis(β-aminoethyl ether)-N,N,N′,N′-tetraacetic acid (EGTA) or diethylenetriamine pentaacetate (DTPA) or like.

In any of the above mentioned embodiments, the anti-PD1 antibody is nivolumab, pembrolizumab, cemiplimab or dosrtalimab.

In any of the above mentioned embodiment, the anti-PDL1 antibody is atezolizumab, avelumab or durvalumab.

In any of the above mentioned embodiments, the concentration of the antibody ranges from 10 mg/ml to 200 mg/ml of the liquid pharmaceutical formulation. In some embodiments, the concentration of the antibody in the formulation is 10 mg/ml, or 25 mg/ml, 30 mg/ml, or 40 mg/ml, or 50 mg/ml, or 60 mg/ml, or 70 mg/ml, or 80 mg/ml, 90 mg/ml, or 100 mg/ml, or 110 mg/ml, or 120 mg/ml, or 130 mg/ml, or 140 mg/ml, 150 mg/ml or 160 mg/ml, or 170 mg/ml or 175 mg/ml or 180 mg/ml or 190 mg/ml or 195 mg/ml or 200 mg/ml.

In any of the above mentioned embodiments, the pH of the disclosed formulation of the present invention is in the range from about 4.5 to about 6.5.

In any of the above mentioned embodiments, the pH of the disclosed formulation of the present invention is in the range from about 5.0 to about 6.0.

In any of the above mentioned embodiments, the pH of the disclosed formulation of the present invention is 6.0±0.2.

In any of the above mentioned embodiments, the anti-PD1/PD-L1 antibody maintains at least 90% of monomeric content of the antibody after storage at 40° C. for two weeks

In any of the above mentioned embodiments, the anti-PD1/PDL1 antibody formulation's osmolality is less than 600 mOsm/kg, preferably less than 300 mOsm/kg.

In another embodiment, the invention discloses a pharmaceutical formulation of an IgG4 anti-PD1 antibody comprising:
i) an IgG4 antibody,
ii) succinate buffer or citrate buffer or acetate buffer, and/or combinations or salts thereof, having pH of about 4.5 to about 6.5
iii) sugar,
iv) a chelating agent or an anti-oxidant or an amino acid and;
v) a surfactant.

In the above embodiment, IgG4 anti-PD1 antibody concentration range from about 10 mg/ml to about 200 mg/ml.

In some embodiments, the concentration of the antibody in the formulation is 10 mg/ml, or 25 mg/ml, 30 mg/ml, or 40 mg/ml, or 50 mg/ml, or 60 mg/ml, or 70 mg/ml, or 80 mg/ml, 90 mg/ml, or 100 mg/ml, or 110 mg/ml, or 120 mg/ml, or 130 mg/ml, or 140 mg/ml, 150 mg/ml or 160 mg/ml, or 170 mg/ml or 175 mg/ml or 180 mg/ml or 190 mg/ml or 195 mg/ml or 200 mg/ml.

In the above mentioned embodiment, the IgG4 anti-PD1 antibody is nivolumab or pembrolizumab.

In another aspect, the invention discloses various methods to control particle formation and aggregation in an IgG4 anti-PD1 antibody composition.

In an embodiment, the invention discloses a method of controlling the formation of sub-visible and visible particles in an IgG4 anti-PD1 antibody composition, wherein the method comprises preparing the antibody composition in succinate buffer or citrate buffer or acetate buffer comprising sugar, an-anti-oxidant or a chelating agent, and surfactant.

In an embodiment, the invention discloses a method of controlling visible_particles formation in an IgG4 anti-PD1 antibody composition, wherein the method comprises preparing the antibody composition in succinate buffer or citrate buffer or acetate buffer composition comprising sugar, a chelating agent, an anti-oxidant, and surfactant.

In an embodiment, the invention discloses a method of controlling the formation of sub-visible and visible particles in nivolumab composition, wherein the method comprises preparing the antibody composition in succinate buffer or citrate buffer or acetate buffer comprising sugar, an-anti-oxidant or a chelating agent, and surfactant.

In an embodiment, the invention discloses a method of controlling visible_particles formation in a nivolumab antibody composition, wherein the method comprises preparing the antibody composition in succinate buffer or citrate buffer or acetate buffer composition comprising sugar, a chelating agent or an anti-oxidant, and surfactant.

In the above embodiment, the visible particles count is reduced to about 10 particles per ml of the antibody composition when stored at 40° C. for two months or at 25° C. for three months or at 2-8° C. for three months.

In an embodiment, the invention discloses a method of controlling sub-visible particles formation of ≥5 μm in size in a nivolumab antibody composition, wherein the method comprises preparation of the antibody composition in succinate buffer or citrate buffer composition comprising sugar, an-anti-oxidant or a chelating agent, and surfactant.

In the above mentioned embodiment, the method controls sub-visible particles formation to less than 1000 particles per ml of the antibody composition, when the formulation is stored at 40° C. for two weeks; and to less than 150 particles per ml of the antibody composition when the antibody composition is stored at 25° C. for three months or at 2-8° C. for three months.

In an embodiment, the invention discloses a method of controlling sub-visible particles formation of ≥10 μm in size in a nivolumab antibody composition, wherein the method comprises preparation of the antibody composition in a succinate buffer composition comprising sugar, an anti-oxidant or a chelating agent, and surfactant.

In the above mentioned embodiment, the sub-visible particles are reduced to less than 200 particles per ml the antibody composition when stored at 40° C. for two weeks and less than 50 particles per ml when stored at room temperature (i.e., 25° C.) for three months.

In an embodiment, the invention discloses a method of controlling sub-visible particles formation of ≥10 μm in size in a nivolumab antibody composition, wherein the method comprises preparation of the antibody composition in histidine-citrate buffer composition comprising sugar, an anti-oxidant, a chelating agent and surfactant.

In the above mentioned embodiment, the sub-visible particles are reduced to less than 100 particles per ml of the antibody composition when stored at 40° C. for two weeks or at 25° C. for three months or at 2-8° C. for three months.

In an embodiment, the invention discloses a method of controlling sub-visible particles formation of ≥25 μm in size in a nivolumab antibody composition, wherein the method comprises preparation of the antibody composition in succinate buffer composition comprising sugar, an anti-oxidant or a chelating agent, and surfactant.

In the above mentioned embodiment, the sub-visible particles are reduced to less than about 25 per ml of the antibody composition when stored at 40° C. for two weeks or at 25° C. for three months or at 2-8° C. for three months.

In an embodiment, the invention discloses a method of controlling sub-visible particles formation of ≥25 μm in size in a nivolumab antibody composition, wherein the method comprises preparation of the antibody composition in succinate buffer composition comprising sugar, an anti-oxidant, a chelating agent and surfactant to the antibody composition.

In the above mentioned embodiment, the sub-visible particles are controlled to less than 10 per ml of antibody composition when stored at 25° C. for three month or at 2-8° C. for three months.

In an embodiment, the invention discloses a method of inhibiting sub-visible particles formation of ≥25 μm in size in a nivolumab antibody composition, wherein the method comprises preparing the antibody composition in succinate buffer composition comprising sugar, an anti-oxidant, a chelating agent and surfactant.

In the above mentioned embodiment, the sub-visible particles count is measured to be zero when the antibody composition is stored at 25° C. for three months.

In an embodiment, the invention discloses a method of controlling sub-visible particles formation of ≥50 μm in size in a nivolumab antibody composition, wherein the method comprises preparation of the antibody composition in succinate buffer composition comprising sugar, an anti-oxidant or chelating agent, and surfactant.

In the above mentioned embodiment, the sub-visible particles are reduced to less than 5 particles per ml of the antibody composition when stored at 40° C. for two weeks or at 25° C. for three months.

In an embodiment, the invention discloses a method of inhibiting sub-visible particles formation of ≥50 μm in size in a nivolumab antibody composition, wherein the method comprises preparation of the antibody composition in succinate buffer composition comprising sugar, an anti-oxidant, a chelating agent and surfactant.

In the above mentioned embodiment, the sub-visible particles count is measured to be zero when the antibody composition is stored at 25° C. for three months.

In any of the above embodiments, the particles are induced by a metal or a chemical or by agitation or a freeze-thaw cycle.

In another embodiment the invention discloses, a method of controlling oxidation of Met₃₄ and Met₈₃ of heavy chain of nivolumab in a pharmaceutical composition of nivolumab, wherein the method comprises preparation of the antibody composition in succinate buffer comprising sugar, a chelating agent, an anti-oxidant and surfactant.

In the above mentioned embodiment, oxidation of methionine residues at 34^{th} and 83^{rd} positions of nivolumab in the antibody formulation prepared in succinate buffer comprising sugar, a chelating agent, an anti-oxidant and surfactant, is controlled better as compared to nivolumab antibody formulation prepared in either succinate buffer comprising sugar, surfactant and antioxidant, but without a chelating agent or succinate buffer comprising sugar, surfactant and chelating agent, but without an antioxidant.

In any of the above mentioned embodiments, the sugar is trehalose or sucrose. The formulation has trehalose in a concentration ranging from 4% to 8% (w/v) and concentration of sucrose is 6% (w/v).

In any of the above mentioned embodiments, the anti-oxidant is methionine.

In the above mentioned embodiment, the concentration of methionine is 10 mM to 20 mM.

In any of the above mentioned embodiments, the chelating agent is diethylenetriamine pentaacetate (DTPA) or ethylenediaminetetraacetic acid (EDTA).

In any of the above mentioned embodiments, the surfactant is polyosrbate-80 or polysorbate-20.

In any of the above mentioned embodiments, the antibody formulations of the invention exhibit stability under at least one of the following conditions, at 40° C. for two weeks, at 25° C. for three months and 2-8° C. for at least three months.

In any of the above embodiments of the invention, the antibody formulation is stable and contains less than 1% of high molecular weight (HMW) species or fragments in the formulation, even after storage under one of the following conditions at 40° C. for two weeks or at 40° C. for one month, or at 40° C. for two months or at 25° C. for one month or at 25° C. for two months or at 25° C. for three months or at 2-8° C. for three months to six months.

In some of the embodiments, nivolumab maintains 90% or more of monomeric content of the antibody after storage under one of the following conditions at 40° C. for two weeks or at 40° C. for one month, or at 40° C. for two months or at 25° C. for one month or at 25° C. for two months or at 25° C. for three months or at 2-8° C. for three months to six months.

In any of the above mentioned embodiments, osmolality of the disclosed antibody formulations is less than 600 mOsm/kg, preferably less than 300 mOsm/kg.

In any of the above mentioned embodiments, the formulation of the antibody is a stable liquid (aqueous) formulation, which can be used for parenteral administration. Parenteral administration includes intravenous, subcutaneous, intra peritoneal, intramuscular administration or any other route of delivery generally considered to be falling under the scope of parenteral administration and as is well known to a skilled person.

In any of the above embodiments of the invention, the stable liquid/aqueous formulation is suitable and can be lyophilized as lyophilized powders. Further, the lyophilized formulation of anti-PD1/PDL1 antibody or an IgG4 antibody can be reconstituted with appropriate diluent to achieve the liquid formulation suitable for administration.

In any of the above mentioned embodiments, the liquid/aqueous anti-PD1/PD-L1 antibody or an IgG4 antibody are compatible with lyophilization process and the lyophilization process does not impact quality attributes of the antibody.

In an embodiment the invention discloses, a liquid pharmaceutical formulation of nivolumab comprising nivolumab, 10-30 mM of succinate buffer or citrate buffer having pH of 5.0 to 6.0, 4% to 8% (w/v) trehalose, 10-30 mM methionine, 0.008 mg/ml of DTPA, 50 to 100 mM sodium chloride and 0.2 mg/ml surfactant, wherein the antibody concentration present in the formulation is in a range of 10 mg/ml to 200 mg/ml.

In the above mentioned embodiment, the surfactant is polysorbate-80 or polysorbate-20.

In another embodiment the invention discloses, a liquid pharmaceutical formulation of pembrolizumab comprising pembrolizumab, 10-30 mM of succinate buffer or acetate buffer having pH of 5.0 to 6.0, 4% to 8% (w/v) trehalose, 0.008 mg/ml of DTPA, and 0.2 mg/ml surfactant, wherein the antibody concentration present in the formulation is in a range of 10 mg/ml to 200 mg/ml

Another aspect of the invention provides a vial, pre-filled syringe or autoinjector device, or any other suitable device comprising any of the subject formulations described herein. In certain embodiments, the aqueous formulation, stored in the vial or pre-filled syringe or an auto injector device comprise anti-PD1/anti-PDL1 antibody or an IgG4 antibody, succinate buffer or acetate buffer or citrate buffer or histidine buffer and/or derivatives or salts or combinations thereof, sugar and surfactant.

Certain specific aspects and embodiments of the invention are more fully described by reference to the following examples. However, these examples should not be construed as limiting the scope of the invention in any manner.

### EXAMPLES

An anti-PD1 antibody, nivolumab, suitable for storage in the present pharmaceutical composition is produced by standard methods known in the art. For example, nivolumab is prepared by recombinant expression of immunoglobulin light and heavy chain genes in a mammalian host cell such as Chinese Hamster Ovary cells. Further, the expressed nivolumab is harvested and the crude harvest is subjected to standard downstream process steps that include purification, filtration and optionally dilution or concentration steps. For example, the crude harvest of nivolumab may be purified using standard chromatography techniques such as affinity chromatography, ion-exchange chromatography and combinations thereof. The purified nivolumab solution can additionally be subjected to one or more filtration steps, and the solution obtained is subjected to further formulation studies.

### Example 1: Assessment of Effect of Various Buffers and Stabilizers on Stability of Nivolumab Formulations

Purified nivolumab antibody approximately 25 mg/ml in various buffer backgrounds such as in histidine/succinate/citrate/acetate buffer background was obtained from downstream chromatographic steps. To know the effect of various buffers and/or stabilizers such as sugar/polyol/amino acid/chelators on the stability of nivolumab, buffer exchange step was performed and the concentration was adjusted to 10 mg/ml. Post which, surfactant polysorbate-80 was added to all the formulation. Nivolumab is approved under the trade name Opdivo® and the currently approved formulation contains 10 mg/ml nivolumab in 20 mM citrate buffer. 3% mannitol, 2.92 mg/mL NaCl, 0.2 mg/mL polysorbate-80 and 0.008 mM DTPA citric acid. Opdivo® formulation has been included in this experiment and denoted as N1 formulation. The final composition of all nivolumab formulations are given in Table 1.

All the samples were measured for their particle formation, opalescence, high molecular weight species using size exclusion chromatography. To measure opalescence, various USP reference opalescence standards were prepared by diluting primary opalescence solution comprising formazin suspension having 4000 NTU ((Nephelometric Turbidity Units). All nivolumab formulations were subjected to accelerated stability studies at 40° C. for four weeks. Post which, the samples were analyzed for low molecular weight (LMW) species and monomer content using size exclusion chromatography (SEC) [results are given in Table 2], charge variants using ion-exchange chromatography (IEX) [results are given in Table 3], particle formation [results are given in Table 4], and opalescence [Table 5]

TABLE 1
Compositions of nivolumab formulations prepared as per example-1

| Sample | |
|---|---|
| Name | Composition |
| N1 | 10 mg/ml nivolumab, 20 mM citrate buffer, 3% mannitol, |
| | 2.92 mg/mL NaCl, 0.2 mg/mL polysorbate 80 and 0.008 |
| | mM DTPA citric acid, pH 6.0. |
| N2 | 10 mg/ml nivolumab, 20 mM succinate phosphate buffer, 3% |
| | Mannitol, 2.92 mg/mL NaCl, 0.2 mg/mL polysorbate 80, pH |
| | 5.8 |
| N3 | 10 mg/ml nivolumab, 20 mM arginine-succinate buffer, 3% |
| | mannitol, 2.92 mg/mL NaCl, 0.2 mg/mL polysorbate 80, pH |
| | 5.8 |
| N4 | 10 mg/ml nivolumab, 20 mM acetate-phosphate, 3% mannitol, |
| | 2.92 mg/mL NaCl, 0.2 mg/mL polysorbate-80, pH 6.1 |
| N5 | 10 mg/ml nivolumab, 20 mM succinate buffer, 3% mannitol, |
| | 2.92 mg/mL NaCl, 0.2 mg/mL polysorbate 80, pH 5.9 |
| N6 | 10 mg/ml nivolumab, 20 mM succinate buffer, 6% trehalose, |
| | 2.92 mg/mL NaCl, 0.2 mg/mL polysorbate 80, pH 5.9 |
| N7 | 10 mg/ml nivolumab, 20 mM succinate buffer, 3% sorbitol, |
| | 2.92 mg/mL NaCl, 0.2 mg/mL polysorbate 80, pH 5.9 |
| N8 | 10 mg/ml nivolumab, 20 mM succinate buffer, 6% sucrose, |
| | 2.92 mg/mL NaC, 1 0.2 mg/mL polysorbate 80, pH 5.9 |
| N9 | 10 mg/ml nivolumab, 20 mM succinate buffer, 3% mannitol, |
| | 0.2 mg/mL polysorbate 80, pH 6.0 |
| N10 | 10 mg/ml nivolumab, 20 mM histidine-citrate buffer, 3% |
| | mannitol, 2.92 mg/mL NaCl, 0.2 mg/mL polysorbate 80, pH |
| | 5.9 |
| N11 | 10 mg/ml nivolumab, 20 mM arginine-citrate buffer, 3% |
| | mannitol, 2.92 mg/mL NaCl, 0.2 mg/mL polysorbate 80, pH |
| | 6.0 |
| N12 | 10 mg/ml nivolumab, 20 mM citrate- [symbol] buffer, 3% |
| | mannitol, 2.92 mg/mL NaCl, 0.2 mg/mL polysorbate 80, pH |
| | 6.0 |
| N13 | 10 mg/ml nivolumab, 20 mM citrate-phosphate buffer, 3% |
| | mannitol, 2.92 mg/mL NaCl, 0.2 mg/mL polysorbate 80, pH |
| | 6.0 |
| N14 | 10 mg/ml nivolumab, 20 mM succinate buffer, 3% mannitol, |
| | 2.92 mg/mL NaCl, 0.05 mg/ml EDTA, 0.2 mg/mL |
| | polysorbate 80, pH 5.9 |
| N15 | 10 mg/ml nivolumab, 20 mM succinate buffer, 3% mannitol, |
| | 2.92 mg/mL NaCl, 0.1 mg/ml EDTA, 0.2 mg/mL polysorbate |
| | 80, pH 5.9 |
| N16 | 10 mg/ml nivolumab, 20 mM histidine buffer, 3% mannitol, |
| | 2.92 mg/mL NaCl, 0.2 mg/mL polysorbate 80, pH 6.0 |
| N17 | 10 mg/ml nivolumab, 20 mM succinate buffer, methionine, |
| | 3% mannitol, 2.92 mg/mL NaCl, 0.2 mg/mL polysorbate 80, |
| | pH 6.0 |

TABLE 2
SEC data of nivolumab formulations prepared as per example-1

| | SEC data at 40° C. | | | | | | | | | | |
|---|---|---|---|---|---|---|---|---|---|---|---|
| Sample | % Monomer at 40° C. | | | | % HMW at 40° C. | | | | % LMW at 40° C. | | |
| name | T0 | T1 W | T2 W | T4 W | T0 | T1 W | T2 W | T4 W | T0 | T2 W | T4 W |
| N1 | 99.3 | 99.2 | 99.2 | 98.9 | 0.6 | 0.7 | 0.7 | 0.9 | 0.09 | 0.1 | 0.2 |
| N2 | 99.6 | 99.3 | 99.2 | 98.9 | 0.4 | 0.6 | 0.8 | 1 | ND | 0.1 | 0.1 |
| N3 | 99.6 | 99.5 | 99.3 | 98.7 | 0.4 | 0.5 | 0.7 | 1.2 | ND | 0.1 | 0.2 |
| N4 | 99.5 | 99.2 | 99.1 | 98.8 | 0.5 | 0.8 | 0.9 | 1.1 | ND | 0.1 | 0.1 |
| N5 | 99.6 | 99.3 | 99.1 | 98.9 | 0.4 | 0.7 | 0.9 | 1 | ND | 0.1 | 0.1 |
| N6 | 99.6 | 99.3 | 99.1 | 98.9 | 0.4 | 0.7 | 0.8 | 1 | ND | 0.04 | 0.1 |
| N7 | 99.6 | 99.3 | 99.1 | 98.7 | 0.4 | 0.7 | 0.9 | 1.1 | ND | 0.1 | 0.1 |
| N8 | 99.6 | 99.3 | 99.2 | 98.9 | 0.4 | 0.6 | 0.8 | 1 | ND | 0.04 | 0.1 |
| N9 | 99.6 | 99.3 | 99.1 | 98.8 | 0.5 | 0.7 | 0.8 | 1.1 | ND | 0.1 | 0.1 |
| N10 | 99.7 | 99.5 | 99.4 | 99.2 | 0.3 | 0.4 | 0.5 | 0.6 | ND | 0.1 | 0.2 |
| N11 | 99.6 | 99.4 | 99.7 | 99.1 | 0.4 | 0.5 | 0.7 | 0.8 | ND | 0.1 | 0.1 |
| N12 | 99.5 | 99.3 | 99.2 | 98.9 | 0.5 | 0.6 | 0.8 | 1 | ND | 0.1 | 0.1 |
| N13 | 99.6 | 99.3 | 99.2 | 98.9 | 0.4 | 0.7 | 0.8 | 1 | ND | 0.1 | 0.1 |
| N14 | 99.6 | 99.4 | 99.2 | 99.1 | 0.4 | 0.6 | 0.7 | 0.8 | ND | 0.1 | 0.1 |
| N15 | 99.6 | 99.4 | 99.3 | 99.1 | 0.4 | 0.6 | 0.7 | 0.8 | ND | 0.1 | 0.1 |
| N16 | 97.4 | 98.8 | 98.1 | 96.6 | 2.6 | 1.2 | 1.8 | 3.2 | ND | 0.1 | 0.2 |
| N17 | 99.6 | 99.4 | 99.3 | 98.9 | 0.4 | 0.5 | 0.6 | 1 | ND | 0.1 | 0.2 |

W—indicates weeks;
ND—not detected

TABLE 3
IEX data of nivolumab formulations prepared as per example-1

| | IEX data at 40° C. | | | | | | | | | | | |
|---|---|---|---|---|---|---|---|---|---|---|---|---|
| Sample | % main peak content | | | | % acidic variants | | | | % basic variants | | | |
| name | T0 | T1 W | T2 W | T4 W | T0 | T1 W | T2 W | T4 W | T0 | T1 W | T2 W | T4 W |
| N1 | 62.8 | 60.1 | 56.0 | 47.4 | 24.3 | 26.7 | 32.0 | 37.7 | 12.8 | 13.1 | 11.9 | 14.8 |
| N2 | 57.3 | 59.1 | 55.2 | 46.8 | 19.4 | 22.2 | 27.6 | 34.2 | 23.2 | 18.6 | 17.1 | 19 |
| N3 | 57.6 | 57.1 | 53.9 | 47.3 | 18.9 | 20.4 | 25.6 | 32 | 23.5 | 22.4 | 20.4 | 20.8 |
| N4 | 57.3 | 59.8 | 55.7 | 48.1 | 19.5 | 22.3 | 27.8 | 33.8 | 23.1 | 17.8 | 16.6 | 18.2 |
| N5 | 57.2 | 57.3 | 54.4 | 47.4 | 19.0 | 21.9 | 27.0 | 33.3 | 23.8 | 20.8 | 18.5 | 19.3 |
| N6 | 57.4 | 57.5 | 54.3 | 47.3 | 19.0 | 21.6 | 26.8 | 33.3 | 23.6 | 20.9 | 18.8 | 19.4 |
| N7 | 57.5 | 56.8 | 53.9 | 46.9 | 19.0 | 21.8 | 27.5 | 34.4 | 23.5 | 21.4 | 18.5 | 18.7 |
| N8 | 57.5 | 57.3 | 54.7 | 47.2 | 18.8 | 21.1 | 26.3 | 32.7 | 23.7 | 21.6 | 19.0 | 20.1 |
| N9 | 57.3 | 57.5 | 54.8 | 47.1 | 19.0 | 21.6 | 27.3 | 33.8 | 23.8 | 20.8 | 17.9 | 19.1 |
| N10 | 57.2 | 56.7 | 53.8 | 47.1 | 19.2 | 21.5 | 26.8 | 32.9 | 23.6 | 21.8 | 21.8 | 20 |
| N11 | 57.4 | 57.5 | 54.3 | 48.2 | 18.9 | 20.3 | 25.4 | 31.4 | 23.7 | 22.2 | 20.3 | 20.4 |
| N12 | 57.9 | 59.8 | 56.1 | 48.4 | 19.1 | 22.7 | 27.6 | 33.4 | 23.0 | 17.5 | 16.3 | 18.2 |
| N13 | 58.1 | 57.7 | 54.9 | 48.3 | 18.6 | 22.1 | 27.1 | 33 | 23.2 | 20.2 | 18.0 | 18.7 |
| N14 | 57.1 | 57.2 | 55.0 | 47.4 | 19.3 | 21.3 | 26.6 | 32.8 | 23.6 | 21.5 | 18.4 | 19.8 |
| N15 | 57.6 | 57.4 | 54.3 | 47.5 | 19.0 | 21.2 | 26.7 | 32.9 | 23.4 | 21.3 | 18.9 | 19.6 |
| N16 | 58.8 | 57.1 | 53.7 | 45.8 | 18.8 | 21.5 | 26.9 | 33.3 | 22.4 | 21.4 | 19.4 | 21 |
| N17 | 57.3 | 59.0 | 54.9 | 48 | 19.1 | 21.2 | 26.5 | 32.4 | 23.6 | 19.8 | 18.5 | 14.8 |

TABLE 4
Measurements of particle formation
in nivolumab formulations

| | Visible particle count | | |
|---|---|---|---|
| Sample | per 1.5 ml at 40° C. | | |
| name | T0 | T2W | T4W |
| N1 | 15 | 38 | 30 |
| N2 | 10 | 15 | 20 |
| N3 | 10 | 25 | 25 |
| N4 | 10 | 13 | 25 |
| N5 | 10 | 20 | 25 |
| N6 | 10 | 20 | 20 |
| N7 | 25 | 15 | 25 |
| N8 | 45 | 21 | 25 |
| N9 | 45 | 16 | 25 |
| N10 | 10 | 20 | 35 |
| N11 | 13 | 15 | 20 |
| N12 | 45 | >50 | 35 |
| N13 | 45 | 20 | 30 |
| N14 | 15 | 13 | 20 |
| N15 | 10 | 25 | 30 |
| N16 | 45 | 25 | >50 |
| N17 | 45 | 25 | 25 |

TABLE 5
Opalescence of nivolumab formulations
prepared as per example 1

| Sample | Opalescence at 40° C. | | | |
|---|---|---|---|---|
| Name | 0 W | 1 W | 2 W | 4 W |
| N1 | ROS II | ROS II | ROS II | ROS II |
| N2 | ROS II | ROS II | ROS II | ROS II |
| N3 | ROS II | ROS II | ROS II | ROS II |
| N4 | ROS II | ROS II | ROS II | ROS II |
| N5 | ROS II | ROS II | ROS II | ROS II |
| N6 | ROS II- | ROS II | ROS II | ROS II |
| | ROS III | | | |
| N7 | ROS II | ROS II | ROS II | ROS II |
| N8 | ROS II | ROS II | ROS II | ROS II |
| N9 | ROS II | ROS II | ROS II | ROS II |
| N10 | ROS II | ROS II | ROS II | ROS II |
| N11 | ROS II | ROS II | ROS II | ROS II |
| N12 | ROS II- | ROS II | ROS II | ROS II |
| | ROS III | | | |
| N13 | ROS II | ROS II | ROS II | ROS II |
| N14 | ROS II | ROS II | ROS II | ROS II |
| N15 | ROS II | ROS II | ROS II | ROS II |
| N16 | ROS II | ROS II | ROS II | ROS II |
| N17 | ROS II | ROS II | ROS II | ROS II |

All the above formulations were also checked for change in pH. It was observed that there is no change in pH of the formulations even after storage for four weeks at 40° C. And all the samples were colorless even after storage at 40° C. for four weeks. Osmolality of all the formulations were found to be less than 350 mOsm/kg.

An IgG4 anti-PD1 antibody, nivolumab, suitable for storage in the present pharmaceutical composition is produced by standard methods known in the art. For example, nivolumab is prepared by recombinant expression of immunoglobulin light and heavy chain genes in a mammalian host cell such as Chinese Hamster Ovary cells. Further, the expressed nivolumab is harvested and the crude harvest is subjected to standard downstream process steps that include purification, filtration and optionally dilution or concentration steps. For example, the crude harvest of nivolumab may be purified using standard chromatography techniques such as affinity chromatography, ion-exchange chromatography and combinations thereof. The purified nivolumab solution can additionally be subjected to one or more filtration steps, and the solution obtained is subjected to further formulation studies.

### Example 2: Effect of Various Buffers and Stabilizers

Purified nivolumab antibody approximately 25 mg/ml in various buffer backgrounds such as in histidine-citrate/succinate/arginine citrate buffer background was obtained from downstream chromatographic steps. Concentration of the antibody was adjusted to 10 mg/ml and subjected to conditions assessing the effect of various buffers and/or stabilizers on the stability of the antibody. Alternatively, nivolumab 10 mg/ml in 20 mM citrate buffer was formulated with nivolumab, 3% mannitol, 2.92 mg/mL NaCl, 0.2 mg/mL polysorbate-80 and 0.008 mM DTPA. The final composition of all nivolumab formulations are given in Table 6.

All the compositions were measured for their visible and sub-visible particles formation, high molecular weight species using size exclusion chromatography before subjecting the samples for accelerated/stress stability conditions. All the formulations were subjected to accelerated stability studies at 40° C. for two weeks to 2 months, and also at room temperature at 25° C. for three months and at 2-8° C. for six months. The samples were then analyzed for high molecular weight (HMW) species, monomer content and low molecular weight (LMW) species using size exclusion chromatography (SEC) [results are given in Table 7 (a) to 7 (c)]. Visible particles [results are given in Table 8] and sub-visible particles were measured by microflow imaging (MFI) technique [results are given in Table 9 (a) to 9 (d)].

TABLE 6
Compositions of formulations prepared as per example-2.

| Sample | |
|---|---|
| Name | Composition |
| F1 | 10 mg/ml nivolumab, 20 mM citrate buffer, 3% mannitol, |
| | 2.92 mg/mL NaCl, 0.2 mg/mL polysorbate 80 and 0.008 mM |
| | DTPA citric acid, pH 6.0. |
| F2 | 10 mg/ml nivolumab, 20 mM arginine-citrate buffer, 6% |
| | trehalose, 10 mM methionine, 0.2 mg/mL polysorbate 80, |
| | pH 5.8 |
| F3 | 10 mg/ml nivolumab, 20 mM succinate buffer, 3% mannitol, |
| | 10 mM methionine, 2.92 mg/ml NaCl 0.2 mg/mL polysorbate |
| | 80, pH 5.8 |
| F4 | 10 mg/ml nivolumab, 20 mM succinate buffer, 6% trehalose, |
| | 10 mM methionine, 2.92 mg/ml NaCl 0.2 mg/mL polysorbate |
| | 80, pH 5.8 |
| F5 | 10 mg/ml nivolumab, 20 mM succinate buffer, 6% trehalose, |
| | 10 mM methionine, 0.05 mg/ml EDTA, 2.92 mg/ml NaCl 0.2 |
| | mg/mL polysorbate 80, pH 5.8 |
| F6 | 10 mg/ml nivolumab, 20 mM succinate, 6% trehalose, 10 mM |
| | methionine, 0.05 mg/ml EDTA, 2.92 mg/ml NaCl, 0.2 mg/mL |
| | polysorbate 80, pH 5.8 |
| F7 | 10 mg/ml nivolumab, 20 mM succinate buffer,4 % trehalose, |
| | 10 mM methionine, 2.92 mg/ml NaCl 0.2 mg/mL polysorbate |
| | 80, pH 5.8 |
| F8 | 10 mg/ml nivolumab, 20 mM succinate buffer, 8% trehalose, |
| | 10 mM methionine, 2.92 mg/ml NaCl 0.2 mg/mL polysorbate |
| | 80, pH 5.8 |
| F9 | 10 mg/ml nivolumab, 20 mM succinate buffer, 6% trehalose, |
| | 0.008 mg/ml DTPA, 2.92 mg/ml NaCl, 0.2 mg/mL |
| | polysorbate 80, pH 5.8 |
| F10 | 10 mg/ml nivolumab, 20 mM succinate buffer, 6% trehalose, |
| | methionine, 0.008 mg/ml, DTPA, 2.92 mg/ml NaCl, 0.2 |
| | mg/mL polysorbate 80, pH 5.8 |
| F11 | 10 mg/ml nivolumab, 20 mM histidine-citrate buffer,8 % |
| | trehalose, 10 mM methionine, 2.92 mg/ml NaCl 0.2 mg/mL |
| | polysorbate 80, pH 5.8 |
| F12 | 10 mg/ml nivolumab, 20 mM histidine-citrate buffer, 6% |
| | trehalose, 0.008 mg/ml DTPA, 2.92 mg/ml NaCl, 0.2 mg/mL |
| | polysorbate 80, pH 5.8 |
| F13 | 10 mg/ml nivolumab, 20 mM histidine-citrate buffer, 6% |
| | trehalose, 10 mM methionine, 2.92 mg/ml NaCl, 0.2 mg/mL |
| | polysorbate 80, pH 5.8 |
| F14 | 10 mg/ml nivolumab, 20 mM histidine-citrate buffer, 6% |
| | trehalose, 0.05 mg/ml EDTA, 2.92 mg/ml NaCl, 0.2 mg/mL |
| | polysorbate 80, pH 5.8 |
| F15 | 10 mg/ml nivolumab, 20 mM histidine-citrate buffer, 4% |
| | trehalose, 10 mM methionine, 2.92 mg/ml NaCl, 0.2 mg/mL |
| | polysorbate 80, pH 5.8 |
| F16 | 10 mg/ml nivolumab, 20 mM histidine-citrate buffer, 6% |
| | trehalose, 10 mM methionine, 0.008 mg/ml DTPA, 2.92 |
| | mg/ml NaCl, 0.2 mg/mL polysorbate 80, pH 5.8 |

TABLE 7(a)
High molecular weight content (i.e., aggregate content) of
formulations prepared as per example-2, measured by SEC.

| | Aggregate content | | | | | | | |
|---|---|---|---|---|---|---|---|---|
| Sample | at 40° C. | | | at 25° C. | | | at 2-8° C. | |
| name | T0 | T1 M | T2 M | T0 | T2 M | T3 M | T3 M | T6 M |
| F1 | 0.2 | 0.8 | 1.2 | 0.2 | 0.4 | 0.5 | 0.3 | 0.3 |
| F2 | 0.3 | 0.8 | 0.8 | 0.3 | 0.5 | 0.6 | 0.4 | 0.5 |
| F3 | 0.3 | 0.8 | 0.8 | 0.3 | 0.5 | 0.6 | 0.4 | 0.4 |
| F4 | 0.3 | 0.8 | 0.8 | 0.3 | 0.5 | 0.5 | 0.3 | 0.5 |
| F5 | 0.4 | 1.0 | 1.1 | 0.4 | 0.6 | 1.0 | 0.5 | 0.6 |
| F6 | 0.3 | 0.8 | 0.9 | 0.3 | 0.5 | 0.6 | 0.3 | 0.4 |
| F7 | 0.2 | 0.7 | 1.2 | 0.2 | 0.4 | 0.4 | 0.2 | 0.3 |
| F8 | 0.2 | 0.6 | 1.0 | 0.2 | 0.3 | 0.4 | 0.2 | 0.2 |
| F9 | 0.2 | 0.6 | 1.1 | 0.2 | 0.5 | 0.4 | 0.2 | 0.3 |
| F10 | 0.2 | 0.5 | 0.9 | 0.2 | 0.3 | 0.3 | 0.3 | 0.2 |
| F11 | 0.3 | 0.5 | 1.2 | 0.3 | 0.3 | 0.3 | 0.3 | 0.3 |
| F12 | 0.3 | 0.5 | 1.2 | 0.3 | 0.3 | 0.3 | 0.3 | 0.3 |
| F13 | 0.2 | 0.6 | 0.6 | 0.2 | 0.3 | 0.4 | 0.3 | 0.3 |
| F14 | 0.3 | 0.6 | 0.7 | 0.3 | 0.4 | 0.3 | 0.3 | 0.4 |
| F15 | 0.3 | 0.5 | 1.3 | 0.3 | 0.4 | 0.4 | 0.3 | 0.3 |
| F16 | 0.3 | 0.5 | 1.1 | 0.3 | 0.3 | 0.3 | 0.3 | 0.3 |

W—indicates weeks,
M—indicates months;
T0—represents data at zero time point

TABLE 7(b)
Percentage monomer content of formulations
prepared as per example-2, measured by SEC.

| | % monomer content | | | | | | | |
|---|---|---|---|---|---|---|---|---|
| Sample | at 40° C. | | | at 25° C. | | | at 2-8° C. | |
| name | T0 | T1 M | T2 M | T0 | T2 M | T3 M | T3 M | T6 M |
| F1 | 99.8 | 99.1 | 98.4 | 99.8 | 99.5 | 99.5 | 99.6 | 99.7 |
| F2 | 99.7 | 99.2 | 99.0 | 99.7 | 99.5 | 99.3 | 99.5 | 99.5 |
| F3 | 99.7 | 99.2 | 99.0 | 99.7 | 99.4 | 99.3 | 99.5 | 99.6 |
| F4 | 99.7 | 99.2 | 99.0 | 99.7 | 99.5 | 99.3 | 99.6 | 99.5 |
| F5 | 99.7 | 98.9 | 98.7 | 99.7 | 99.3 | 98.9 | 99.4 | 99.4 |
| F6 | 99.7 | 99.2 | 99.0 | 99.7 | 99.5 | 99.3 | 99.6 | 99.6 |
| F7 | 99.8 | 99.2 | 98.5 | 99.8 | 99.6 | 99.5 | 99.7 | 99.7 |
| F8 | 99.8 | 99.3 | 98.8 | 99.8 | 99.6 | 99.5 | 99.7 | 99.7 |
| F9 | 99.8 | 99.2 | 98.6 | 99.8 | 99.5 | 99.5 | 99.6 | 99.7 |
| F10 | 99.8 | 99.4 | 98.9 | 99.8 | 99.7 | 99.6 | 99.7 | 99.8 |
| F11 | 99.7 | 99.4 | 99.0 | 99.7 | 99.6 | 99.6 | 99.6 | 99.7 |
| F12 | 99.7 | 99.3 | 98.5 | 99.7 | 99.6 | 99.6 | 99.6 | 99.7 |
| F13 | 99.8 | 99.4 | 99.2 | 99.8 | 99.6 | 99.5 | 99.7 | 99.6 |
| F14 | 99.8 | 99.3 | 99.1 | 99.8 | 99.6 | 99.3 | 99.6 | 99.6 |
| F15 | 99.7 | 99.3 | 98.4 | 99.7 | 99.6 | 99.6 | 99.6 | 99.7 |
| F16 | 99.7 | 99.4 | 98.7 | 99.7 | 99.6 | 99.6 | 99.6 | 99.7 |

M—indicates months;
T0—represents data at zero time point

TABLE 7(c)
Low molecular weight content (i.e., LMW content) of formulations
prepared as per example-2, measured by SEC.

| | % LMW content | | | | | | | |
|---|---|---|---|---|---|---|---|---|
| Sample | at 40° C. | | | at 25° C. | | | at 2-8° C. | |
| name | T0 | T1 M | T2 M | T0 | T2 M | T3 M | T3 M | T6 M |
| F1 | ND | 0.13 | 0.3 | ND | 0.1 | 0.1 | 0.1 | 0.0 |
| F2 | ND | 0.1 | 0.17 | ND | 0.07 | 0.1 | 0.2 | 0.0 |
| F3 | ND | 0.1 | 0.16 | ND | 0.07 | 0.1 | 0.1 | 0.0 |
| F4 | ND | 0.1 | 0.18 | ND | 0.07 | 0.2 | 0.1 | 0.0 |
| F5 | ND | 0.1 | 0.17 | ND | 0.06 | 0.1 | 0.1 | 0.0 |
| F6 | ND | 0.1 | 0.16 | ND | 0.05 | 0.1 | 0.1 | 0.0 |
| F7 | ND | 0.12 | 0.3 | ND | 0.1 | 0.1 | 0.1 | 0.0 |
| F8 | ND | 0.11 | 0.3 | ND | 0.1 | 0.1 | 0.1 | 0.0 |
| F9 | ND | 0.13 | 0.2 | ND | 0.1 | 0.1 | 0.1 | 0.0 |
| F10 | ND | 0.13 | 0.3 | ND | 0.1 | 0.1 | 0.1 | 0.0 |
| F11 | ND | 0.14 | 0.3 | ND | 0.0 | 0.1 | 0.1 | 0.0 |
| F12 | ND | 0.13 | 0.2 | ND | 0.1 | 0.1 | 0.1 | 0.0 |
| F13 | ND | 0.1 | 0.18 | ND | 0.05 | 0.1 | 0.1 | 0.0 |
| F14 | ND | 0.1 | 0.18 | ND | 0.06 | 0.3 | 0.1 | 0.0 |
| F15 | ND | 0.14 | 0.2 | ND | 0.1 | 0.1 | 0.1 | 0.0 |
| F16 | ND | 0.15 | 0.3 | ND | 0.1 | 0.1 | 0.1 | 0.0 |

W—indicates weeks, M—indicates months; T0—represents data at zro time point; ND—Not detected.

TABLE 8
Visible particles count of formulations, prepared as per example-2.

| | Particles count (≥100 μm) | | | | | | | |
|---|---|---|---|---|---|---|---|---|
| Sample | at 40° C. | | | at 25° C. | | | At 2-8° C. | |
| name | T0 | T1 M | T2 M | T0 | T2 M | T3 M | T0 | T3 M |
| F1 | 8 | 10 | 9 | 8 | 11 | 13 | 8 | 9 |
| F2 | 8 | 11 | 9 | 8 | 25 | 11 | 8 | 8 |
| F3 | 8 | 11 | 8 | 8 | 3 | 5 | 8 | 8 |
| F4 | 8 | 12 | 9 | 8 | 7 | 4 | 8 | 6 |
| F5 | 8 | 11 | 9 | 8 | 3 | 8 | 8 | 6 |
| F6 | 8 | 11 | 13 | 8 | 10 | 11 | 8 | 8 |
| F7 | 18 | 18 | 13 | 18 | 14 | 14 | 18 | 7 |
| F8 | 16 | 18 | 13 | 16 | 12 | 9 | 16 | 8 |
| F9 | 18 | 5 | 9 | 18 | 7 | 9 | 18 | 5 |
| F10 | 16 | 5 | 6 | 16 | 7 | 5 | 16 | 4 |
| F11 | 6 | 4 | 5 | 6 | 5 | 5 | 6 | 4 |
| F12 | 6 | 3 | 4 | 6 | 8 | 4 | 6 | 4 |
| F13 | 8 | 10 | 9 | 8 | 5 | 10 | 8 | 10 |
| F14 | 8 | 10 | 11 | 8 | 9 | 11 | 8 | 10 |
| F15 | 8 | 2 | 2 | 8 | 6 | 5 | 8 | 4 |
| F16 | 6 | 3 | 4 | 6 | 6 | 4 | 6 | 6 |

M—indicates months;
T0—represents data at zero time point

TABLE 9(a)
Sub visible particles count with size ≥5 μm of formulations,
prepared as per example-2 and measured by MFI.

| | Sub visible particles count | | | | | | |
|---|---|---|---|---|---|---|---|
| Sample | at 40° C. | | at 25° C. | | | at 2-8° C. | |
| name | T0 | T2 W | T0 | T2 M | T3 M | T0 | T3 M |
| F1 | 1124 | 1324 | 1124 | 291 | 281 | 1124 | 437 |
| F2 | 173 | 1445 | 173 | 300766 | 117 | 173 | 173 |
| F3 | 54 | 869 | 54 | 328 | 166 | 54 | 54 |
| F4 | 24 | 1136 | 24 | 222 | 93 | 24 | 24 |
| F5 | 44 | 533 | 44 | 209 | 4197 | 44 | 44 |
| F6 | 111 | 646 | 111 | 226903 | 102 | 111 | 111 |
| F7 | 145 | 1447 | 145 | 1223 | 379 | 145 | 248 |
| F8 | 230 | 1127 | 230 | 372 | 81 | 230 | 146 |
| F9 | 82 | 577 | 82 | 157 | 365 | 82 | 109 |
| F10 | 90 | 774 | 90 | 85 | 52 | 90 | 118 |
| F11 | 92 | 1187 | 92 | 29 | 60 | 92 | 406 |
| F12 | 436 | 2194 | 436 | 38 | 86 | 436 | 210 |
| F13 | 144 | 1136 | 144 | 291 | 156 | 144 | 144 |
| F14 | 170 | 1162 | 170 | 662 | 14080 | 170 | 170 |
| F15 | 56 | 774 | 56 | 29 | 26 | 56 | 29 |
| F16 | 143 | 1309 | 143 | 230 | 104 | 143 | 201 |

W—indicates weeks,
M—indicates months;
T0—represents data at zero time point

TABLE 9(b)
Sub visible particles size of ≥10 μm of formulations,
prepared as per example-2 and measured by MFI.

| | Sub visible particles count | | | | | | |
|---|---|---|---|---|---|---|---|
| Sample | at 40° C. | | at 25° C. | | | at 2-8° C. | |
| name | T0 | T2 W | T0 | T2 M | T3 M | T0 | T3 M |
| F1 | 228 | 224 | 228 | 94 | 80 | 228 | 170 |
| F2 | 35 | 353 | 35 | 30196 | 29 | 35 | 35 |
| F3 | 24 | 232 | 24 | 106 | 67 | 24 | 24 |
| F4 | 15 | 198 | 15 | 119 | 83 | 15 | 15 |
| F5 | 9 | 172 | 9 | 61 | 2196 | 9 | 9 |
| F6 | 27 | 155 | 27 | 28821 | 35 | 27 | 27 |
| F7 | 41 | 276 | 41 | 343 | 146 | 41 | 87 |
| F8 | 97 | 181 | 97 | 168 | 26 | 97 | 78 |
| F9 | 20 | 34 | 20 | 44 | 78 | 20 | 37 |
| F10 | 39 | 112 | 39 | 29 | 12 | 39 | 49 |
| F11 | 18 | 413 | 18 | 6 | 17 | 18 | 127 |
| F12 | 67 | 645 | 67 | 9 | 23 | 67 | 63 |
| F13 | 54 | 293 | 54 | 95 | 36 | 54 | 54 |
| F14 | 63 | 327 | 63 | 117 | 7089 | 63 | 63 |
| F15 | 15 | 250 | 15 | 6 | 11 | 15 | 12 |
| F16 | 47 | 86 | 47 | 35 | 29 | 47 | 66 |

W—indicates weeks,
M—indicates months;
T0—represents data at zero time point

TABLE 9(c)
Sub visible particles size of ≥25 μm of formulations,
prepared as per example-2, and measured by MFI.

Sub visible particles count

| | | | |
|---|---|---|---|
| Sample | at 40° C. | at 25° C. | at 2-8° C. |

| name | T0 | T2 W | T0 | T2 M | T3 M | T0 | T3 M |
|---|---|---|---|---|---|---|---|
| F1 | 29 | 26 | 29 | 6 | 26 | 29 | |
| F2 | 3 | 52 | 3 | 34 | 3 | 3 | 3 |
| F3 | 12 | 52 | 12 | 30 | 12 | 12 | 12 |
| F4 | 0 | 9 | 0 | 25 | 14 | 0 | 0 |
| F5 | 6 | 0 | 6 | 16 | 22 | 6 | 6 |
| F6 | 6 | 9 | 6 | 1905 | 9 | 6 | 6 |
| F7 | 9 | 9 | 9 | 51 | 37 | 9 | 29 |
| F8 | 30 | 26 | 30 | 30 | 12 | 30 | 14 |
| F9 | 3 | 0 | 3 | 6 | 14 | 3 | 12 |
| F10 | 9 | 17 | 9 | 9 | 0 | 9 | 9 |
| F11 | 0 | 34 | 0 | 3 | 0 | 0 | 9 |
| F12 | 9 | 52 | 9 | 0 | 9 | 9 | 9 |
| F13 | 9 | 26 | 9 | 18 | 6 | 9 | 9 |
| F14 | 13 | 17 | 13 | 22 | 1421 | 13 | 13 |
| F15 | 0 | 9 | 0 | 3 | 3 | 0 | 3 |
| F16 | 6 | 0 | 6 | 6 | 6 | 6 | 6 |

W-indicates weeks,
M-indicates months;
T0-represents data at zero time point

TABLE 9(d)
Sub visible particles size of ≥50 μm of formulations,
prepared as per example-2, and measured by MFI.

Sub visible particles count

| | | | |
|---|---|---|---|
| Sample | at 40° C. | at 25° C. | at 2-8° C. |

| name | T0 | T2 W | T0 | T2 M | T3 M | T0 | T3 M |
|---|---|---|---|---|---|---|---|
| F1 | 9 | 17 | 9 | 9 | 3 | 9 | 6 |
| F2 | 0 | 9 | 0 | 3 | 3 | 0 | 0 |
| F3 | 6 | 0 | 6 | 3 | 3 | 6 | 6 |
| F4 | 0 | 0 | 0 | 3 | 3 | 0 | 0 |
| F5 | 0 | 0 | 0 | 3 | 6 | 0 | 0 |
| F6 | 3 | 0 | 3 | 265 | 3 | 3 | 3 |
| F7 | 3 | 0 | 3 | 3 | 3 | 3 | 3 |
| F8 | 3 | 9 | 3 | 3 | 3 | 3 | 0 |
| F9 | 0 | 0 | 0 | 0 | 6 | 0 | 0 |
| F10 | 3 | 0 | 3 | 0 | 0 | 3 | 0 |
| F11 | 0 | 0 | 0 | 0 | 0 | 0 | 3 |
| F12 | 0 | 0 | 0 | 0 | 0 | 0 | 3 |
| F13 | 6 | 0 | 6 | 3 | 3 | 6 | 6 |
| F14 | 3 | 0 | 3 | 6 | 173 | 3 | 3 |
| F15 | 0 | 0 | 0 | 0 | 0 | 0 | 0 |
| F16 | 3 | 0 | 3 | 3 | 3 | 3 | 0 |

W-indicates weeks,
M-indicates months;
T0-represents data at zero time point

### Example-3: Stability of Nivolumab Antibody Formulations Under Various Stress Conditions

Based on the above data, some of the formulations of example-1, viz., F1 control, F9, F10, F12 and F16 were further subjected for agitation, freeze/thaw, chemical oxidation and metal induced oxidation stress to know the impact of these conditions on the stability of the formulations.

### a) Agitation Study:

As mentioned above, all five samples of example-3 were subjected for agitation under 300 RPM for four days at 25° C. The samples were then measured for visible particles and sub-visible particles by MFI, Results are given in Table 10, and Table 11.

TABLE 10
Visible particles data of agitation
induced stress study formulations

| Sample | | | | | |
|---|---|---|---|---|---|
| name | T0 | T1D | T2D | T3D | T4D |
| F1 | 15 | 25 | 25 | 35 | 35 |
| F9 | 15 | 25 | 25 | 35 | 35 |
| F10 | 15 | 25 | 25 | 35 | 35 |
| F12 | 15 | 25 | 25 | 35 | 35 |
| F16 | 15 | 15 | 25 | 35 | 35 |

D-indicates Days;
T0-represents data at zero time point

TABLE 11
Sub-visible particles data of induced stress study samples, measured by MFI.

| Sample | ≥5 μm | | | ≥10 μm | | | ≥25 μm | | | ≥50 μm | | |
|---|---|---|---|---|---|---|---|---|---|---|---|---|
| name | T0 | T2 D | T4 D | T0 | T2 D | T4 D | T0 | T2 D | T4 D | T0 | T2 D | T4 D |
| F1 | 84 | 552 | 1373 | 21 | 69 | 132 | 6 | 3 | 12 | 0 | 3 | 0 |
| F9 | 115 | 1029 | 971 | 15 | 58 | 127 | 3 | 3 | 6 | 0 | 0 | 0 |
| F10 | 101 | 624 | 1183 | 17 | 59 | 166 | 0 | 0 | 9 | 0 | 0 | 0 |
| F12 | 116 | 1025 | 830 | 24 | 94 | 63 | 9 | 3 | 0 | 0 | 0 | 0 |
| F16 | 139 | 543 | 648 | 9 | 44 | 58 | 0 | 0 | 3 | 0 | 0 | 0 |

D-indicates Days;
T0-represents data at zero time point

### b) Freeze-Thaw Study

All five samples of Example-3 were also further subjected for five free-thaw cycles and in each freeze-thaw cycle samples were frozen at 80° C. for 24 hours and thawed at room temperature. Post five freeze-thaw cycles, samples were measured for visible particles, sub-visible particles by MFI, and High molecular weight species and monomer content by Size exclusion chromatography. Results are given in below Table 12, Table 13 and Table 14. It has been observed that, F1-control sample precipitated after four freeze-thaw cycles.

TABLE 12
Visible particles data of samples after multiple freeze-thaw cycles.

| Sample name | T0 | T1FT | T2FT | T3FT | T4FT | T5FT |
|---|---|---|---|---|---|---|
| F1 | 15 | 25 | 25 | 35 | 35 | — |
| F9 | 15 | 25 | 25 | 35 | 35 | 45 |
| F10 | 15 | 25 | 25 | 35 | 35 | 45 |
| F12 | 15 | 25 | 25 | 35 | 35 | 45 |
| F16 | 15 | 25 | 25 | 35 | 35 | 45 |

FT-indicates Freeze-thaw cycle;
T0-represents data at zero time point

TABLE 13
Sub-visible particles data of samples after
multiple freeze-thaw cycles, measured by MFI.

| Sample | ≥5 μm | | | ≥ 10 μm | | | ≥ 25 μm | | | ≥ 50 μm | | |
|---|---|---|---|---|---|---|---|---|---|---|---|---|
| name | T0 | T1FT | T5FT | T0 | T1FT | T5FT | T0 | T1FT | T5FT | T0 | T1FT | T5FT |
| F1 | 84 | 95 | 398 | 21 | 24 | 85 | 6 | 9 | 0 | 0 | 0 | 0 |
| F9 | 115 | 45 | 126 | 15 | 18 | 21 | 3 | 3 | 3 | 0 | 3 | 0 |
| F10 | 101 | 12 | 137 | 17 | 0 | 32 | 0 | 0 | 3 | 0 | 0 | 0 |
| F12 | 116 | 148 | 609 | 24 | 41 | 84 | 9 | 9 | 0 | 0 | 0 | 0 |
| F16 | 139 | 87 | 46 | 9 | 9 | 6 | 0 | 0 | 3 | 0 | 0 | 0 |

FT-indicates Freeze-thaw cycle;
T0-represents data at zero time point

TABLE 14
Aggregate content and monomer content of samples after
multiple freeze-thaw cycles, measured by SEC.

| Sample | % HMW | | | | | | % monomer | | | | | |
|---|---|---|---|---|---|---|---|---|---|---|---|---|
| name | T0 | T1FT | T2FT | T3FT | T4FT | T5FT | T0 | T1FT | T2FT | T3FT | T4FT | T5FT |
| F1 | 0.3 | 0.5 | 0.8 | 1.0 | 1.3 | 1.4 | 99.7 | 99.5 | 99.2 | 99.0 | 98.7 | 98.7 |
| F9 | 0.2 | 0.2 | 0.3 | 0.3 | 0.3 | 0.3 | 99.7 | 99.8 | 99.7 | 99.8 | 99.8 | 99.7 |
| F10 | 0.2 | 0.3 | 0.3 | 0.3 | 0.3 | 0.3 | 99.8 | 99.7 | 99.7 | 99.7 | 99.8 | 99.7 |
| F12 | 0.2 | 0.2 | 0.2 | 0.2 | 0.2 | 0.2 | 99.8 | 99.8 | 99.8 | 99.8 | 99.8 | 99.8 |
| F16 | 0.2 | 0.2 | 0.2 | 0.2 | 0.2 | 0.2 | 99.8 | 99.8 | 99.8 | 99.8 | 99.8 | 99.8 |

FT-indicates Freeze-thaw cycle;
T0-represents data at zero time point

### c) Chemical Oxidation Study:

All five samples of Example-3 were further subjected for chemical oxidation with 0.1% hydrogen peroxide (H₂O₂) and 1% H₂O₂ and samples were kept at 25° C. for three days. Samples were then measured for visible particles, sub-visible particles by MFI, monomer and aggregate content by SEC. Results of the study are given in Table 15, Table 16, and Table 17.

TABLE 15
Visible particles data of samples prepared as per Example-3,
after chemical induced oxidation stress study.

| Sample | 0.1% H₂O₂ | | | 1% H₂O₂ | | |
|---|---|---|---|---|---|---|
| name | T0 | T1 D | T3 D | T0 | T1 D | T3 D |
| F1 | 25 | 25 | 25 | 70 | 45 | 73 |
| F9 | 25 | 25 | 25 | 45 | 45 | 80 |
| F10 | 25 | 25 | 25 | 45 | 45 | 55 |
| F12 | 25 | 25 | 15 | 45 | 45 | 55 |
| F16 | 25 | 25 | 25 | 45 | 45 | 55 |

D-indicates Days;
T0-represents data at zero time point

TABLE 16 (a)
Sub-visible particles data of samples prepared as per
Example-2, after 0.1% H₂O₂ chemical induced oxidation

| Sample | ≥5 μm | | ≥10 μm | | ≥25 μm | | ≥50 μm | |
|---|---|---|---|---|---|---|---|---|
| name | T0 | T3 D | T0 | T3 D | T0 | T3 D | T0 | T3 D |
| F1 | 123 | 100 | 29 | 37 | 0 | 9 | 0 | 0 |
| F9 | 320 | 282 | 101 | 57 | 3 | 9 | 0 | 0 |
| F10 | 82 | 116 | 32 | 13 | 6 | 3 | 3 | 0 |
| F12 | 46 | 127 | 9 | 26 | 0 | 3 | 0 | 3 |
| F16 | 45 | 54 | 3 | 12 | 3 | 0 | 0 | 0 |

D-indicates Days;
T0-represents data at zero time point

TABLE 16 (b)
Sub-visible particles data of samples prepared as per
Example-3, after 1% H₂O₂ chemical induced oxidation

| Sample | ≥5 μm | | ≥10 μm | | ≥25 μm | | ≥50 μm | |
|---|---|---|---|---|---|---|---|---|
| name | T0 | T3 D | T0 | T3 D | T0 | T3 D | T0 | T3 D |
| F1 | 161 | 99 | 13 | 48 | 3 | 24 | 0 | 0 |
| F9 | 149 | 97 | 10 | 25 | 0 | 0 | 0 | 0 |
| F10 | 104 | 254 | 6 | 41 | 0 | 0 | 0 | 0 |
| F12 | 58 | 112 | 12 | 14 | 0 | 9 | 0 | 9 |
| F16 | 186 | 35 | 15 | 12 | 0 | 6 | 0 | 0 |

D-indicates Days;
T0-represents data at zero time point

TABLE 17
HMW and monomer content of samples prepared as per Example-3, after chemical
induced oxidation stress with 0.1% H₂O₂ and 1% H₂O₂, measured by SEC.

| | % HMW | | | | | | % monomer | | | | | |
|---|---|---|---|---|---|---|---|---|---|---|---|---|
| Sample | 0.1% H₂O₂ | | | 1% H₂O₂ | | | 0.1% H₂O₂ | | | 1% H₂O₂ | | |
| name | T0 | T1 D | T3 D | T0 | T1 D | T3 D | T0 | T1 D | T3 D | T0 | T1 D | T3 D |
| F1 | 0.7 | 1.0 | 1.3 | 1.3 | 1.2 | 1.7 | 99.3 | 99.0 | 98.6 | 97.9 | 98.5 | 98.1 |
| F9 | 0.2 | 0.2 | 0.3 | 0.3 | 4.3 | 0.6 | 99.8 | 99.8 | 99.6 | 99.6 | 99.5 | 99.2 |
| F10 | 0.2 | 0.2 | 0.3 | 0.3 | 1.8 | 0.5 | 99.8 | 99.8 | 99.6 | 99.7 | 99.5 | 99.3 |
| F12 | 0.2 | 0.2 | 0.2 | 0.2 | 0.3 | 0.4 | 99.8 | 99.8 | 99.7 | 99.6 | 99.5 | 99.4 |
| F16 | 0.2 | 0.2 | 0.2 | 0.2 | 0.2 | 0.5 | 99.8 | 99.9 | 99.8 | 99.7 | 99.6 | 99.3 |

D-indicates Days;
T0-represents data at zero time point

### Metal Induced Oxidation Study:

All five samples of Example-3 were further subjected for metal induced oxidation with 0.0007 mg/ml of Cobalt and samples were kept at 25° C. for three days. Post which, samples were measured for visible particles, sub-visible particles by MFI, monomer and aggregate content by SEC. Results of the study are given in Table 13, Table 14, and Table 15.

TABLE 18
Visible particles data of metal
induced oxidation stress study
samples prepared as per Example-3.

| Sample | | | |
|---|---|---|---|
| name | T0 | T1D | T3D |
| F1 | 25 | 25 | 25 |
| F9 | 25 | 25 | 25 |
| F10 | 25 | 25 | 25 |
| F12 | 25 | 25 | 25 |
| F16 | 25 | 25 | 25 |

D-indicates Days;
T0-represents data at zero time point

TABLE 19
Sub-visible particles data metal induced
oxidation study of samples prepared
as per Exmaple-3, measured by MFI.

| Sample | ≥5 μm | | ≥10 μm | | ≥25 μm | | ≥50 μm | |
|---|---|---|---|---|---|---|---|---|
| name | T0 | T3D | T0 | T3D | T0 | T3D | T0 | T3D |
| F1 | 269 | 193 | 50 | 26 | 3 | 0 | 0 | 0 |
| F9 | 149 | 220 | 20 | 57 | 0 | 3 | 0 | 0 |
| F10 | 41 | 256 | 3 | 39 | 0 | 3 | 0 | 0 |
| F12 | 133 | 130 | 19 | 35 | 3 | 6 | 0 | 0 |
| F16 | 109 | 515 | 15 | 111 | 0 | 3 | 0 | 0 |

D-indicates Days;
T0-represents data at zero time point

TABLE 20
HMW and monomer content of metal induced oxidation stress
study samples prepared as per Example-3, measured by SEC.

| Sample | % HMW | | | % monomer | | |
|---|---|---|---|---|---|---|
| name | T0 | T1 D | T3 D | T0 | T1 D | T3 D |
| F1 | 0.4 | 0.7 | 0.7 | 99.6 | 99.2 | 99.3 |
| F9 | 0.2 | 0.3 | 0.3 | 99.8 | 99.8 | 99.8 |
| F10 | 0.2 | 0.3 | 0.3 | 99.8 | 99.8 | 99.7 |
| F12 | 0.2 | 0.2 | 0.2 | 99.9 | 99.8 | 99.8 |
| F16 | 0.2 | 0.2 | 0.2 | 99.9 | 99.8 | 99.8 |

D-indicates Days;
T0-represents data at zero time point

### Example-4: Oxidation Study of Nivolumab Antibody Formulations

Nivolumab samples of example-1 viz., F4, F9, F10, F12, F13 and F16 were stored at 40° C. for two months. The samples were then subjected to liquid chromatography-mass spectrometry and measured the oxidation levels at various point. Oxidation data of control sample is measured at zero time point (T0) without being subjected to storage at specific temperature condition. Results of the oxidation are given in Table 21.

TABLE 21
Percentage methionine oxidation of samples of Example-4.

| | % Met oxidation | | | | |
|---|---|---|---|---|---|
| Sample | AA | AA | AA | AA | AA |
| name | (24-38) | (77-87) | (242-248) | (338-353) | (410-432) |
| F1 | 3.93 | 2.89 | 5.75 | 4.71 | 2.39 |
| F4 | 7.49 | 5.83 | 5.31 | 5.45 | 4.17 |
| F8 | 5.89 | 4.56 | 5.43 | 4.43 | 4.03 |
| F9 | 4.01 | 3.09 | 4.72 | 4.41 | 2.97 |
| F13 | 3.7 | 2.81 | 4.24 | 3.58 | 2.56 |
| F12 | 3.7 | 2.81 | 6.56 | 4.85 | 3.54 |
| F16 | 3.78 | 2.87 | 6.22 | 3.85 | 2.65 |

All the above formulations were also checked for change in pH. It was observed that there is no change in pH of the formulations even after storage under accelerated conditions. And all the samples were colorless and osmolality of all the formulations were found to be less than 350 mOsm/kg under agitation induced stress study, freeze-thaw stress study and metal induced stress study.

### Example 5: High Concentration Anti-PD1 Antibody Formulations

Nivolumab 10 mg/ml in succinate buffer, comprising 60 mg/ml trehalose, methionine, 2.92 mg/ml sodium chloride, 0.008 mg/ml DTPA and 0.2 mg/ml polysorbate-80 were further concentrated up to 150 mg/ml by ultrafiltration. Alternatively, this high concentration nivolumab sample buffer was buffer exchanged into acetate buffer. Post which, these two high concentration nivolumab samples in succinate buffer and in acetate buffer were subjected for stress stability condition at 40° C. for one week and for 5 days respectively and measured for high molecular weight species, monomer content and low molecular weight species using SEC. Further, acidic variants and main peak contents of the samples were measured using IEX chromatography and viscosity of the samples were measured using viscometer. Results of the study are given below in Table 22.

TABLE 22
Composition of high concentration nivolumab formulation prepared
as per Example-5, and quality attributes of the formulations

| | | | % of | | | % of | | % of | |
|---|---|---|---|---|---|---|---|---|---|
| | % HMW | | monomer | | | acidic | | main peak | |
| | content at | | content at | | Viscosity | variants at | | content at | |
| Sample | 40° C. | | 40° C. | | (cP) | 40° C. | | 40° C. | |
| composition | T0 | T1 W | T0 | T1 W | T0 | T0 | T5 D | T0 | T5 D |
| 155 mg/ml | 0.8 | 1.4 | 99.2 | 99.5 | 3.3 | 13.8 | 18.0 | 53.1 | 52.6 |
| nivolumab, 20 | | | | | | | | | |
| mM succinate | | | | | | | | | |
| buffer, 60 mg/ml | | | | | | | | | |
| trehalose, 10 | | | | | | | | | |
| mMmethionine, | | | | | | | | | |
| 0.008 mg/ml | | | | | | | | | |
| DTPA 0.2 mg/ml | | | | | | | | | |
| polysorbate | | | | | | | | | |
| 133 mg/ml | 0.9 | 1.4 | 99.1 | 98.6 | NM | 12.6 | 11.6 | 60.2 | 58.6 |
| nivolumab, 20 | | | | | | | | | |
| mM acetate | | | | | | | | | |
| buffer, 60 mg/ml | | | | | | | | | |
| trehalose, 10 | | | | | | | | | |
| mMmethionine, | | | | | | | | | |
| 0.008 mg/ml | | | | | | | | | |
| DTPA and 0.2 | | | | | | | | | |
| mg/ml | | | | | | | | | |
| polysorbate | | | | | | | | | |

D—indicates Days; T0—represents data at zero time point; W—indicates week

### Example 6: Stability of Other Anti-PD1 Antibody Formulations

Another anti-PD1 antibody, pembrolizumab expressed in CHO cells and the expressed antibody has been purified by techniques already known in the art. 35 mg/ml of purified pembrolizumab obtained from downstream chromatographic step, was subjected for buffer exchange step with succinate or histidine acetate buffer. In addition, pembrolizumab in acetate buffer obtained from downstream chromatographic technique maintained as it as. To all the pembrolizumab antibody samples in various buffers, combination of various excipients such as sugars, amino acid, chelating agents and surfactant were added. Composition of all pembrolizumab samples are given in Table 23.

Post which, these samples were subjected for accelerated stability studies at 40° C. for one month and various quality attributes of the samples such as change in pH, osmolality, high molecular weight content, monomer content and low molecular weight content using SEC and charge variants using IEX were measured. Further, opalescence of the samples were measured.

Results of the study are given in Table 24-26.

TABLE 23
Compositions of pembrolizumab formulations
prepared as per example-6

| Sample | |
|---|---|
| Name | Composition |
| P1 | 25 mg/ml pembrolizumab, 20 mM acetate buffer, 7% |
| | sucrose and polysorbate-80 |
| P2 | 25 mg/ml pembrolizumab, 10 mM acetate buffer, 4.5% |
| | trehalose glycine, and polysorbate-80 |
| P3 | 25 mg/ml pembrolizumab, 10 mM acetate buffer, 4.5% |
| | trehalose proline, and polysorbate-80 |
| P4 | 25 mg/ml pembrolizumab, 10 mM acetate buffer, 10% |
| | trehalose and polysorbate-80 |
| P5 | 25 mg/ml pembrolizumab, 10 mM acetate buffer, 10% |
| | trehalose, DTPA and polysorbate-80 |
| P6 | 25 mg/ml pembrolizumab, 10 mM acetate buffer, 10% |
| | trehalose, DTPA, 60 mM arginine and polysorbate-80 |
| P7 | 25 mg/ml pembrolizumab, 10 mM acetate buffer, 10% |
| | asorbitol nd polysorbate-80 |
| P8 | 25 mg/ml pembrolizumab, 10 mM acetate buffer, 12% |
| | sucrose and polysorbate-80 |
| P9 | 25 mg/ml pembrolizumab, 10 mM histidine-acetate |
| | buffer, 10% trehalose and polysorbate-80 |
| P10 | 25 mg/ml pembrolizumab, 10 mM succinate buffer, 4.5% |
| | trehalose glycine, and polysorbate-80 |
| P11 | 25 mg/ml pembrolizumab, 10 mM succinate buffer, 4.5% |
| | trehalose proline, and polysorbate-80 |
| P12 | 25 mg/ml pembrolizumab, 10 mM succinate buffer, 10% |
| | trehalose and polysorbate-80 |
| P13 | 25 mg/ml pembrolizumab, 10 mM succinate buffer, 10% |
| | trehalose, DTPA and polysorbate-80 |
| P14 | 25 mg/ml pembrolizumab, 10 mM succonate buffer, 10% |
| | trehalose, DTPA, 60 mM arginine and polysorbate-80 |
| P15 | 25 mg/ml pembrolizumab, 10 mM succinate buffer, 10% |
| | sorbitol, and polysorbate-80 |
| P16 | 25 mg/ml pembrolizumab, 10 mM succinate buffer, 12% |
| | sucrose, and polysorbate-80 |

TABLE 24
Various quality attributes of pembrolizumab
formulations prepared as per Example-6

| | pH | | Osmolality (mOsm/Kg) | |
|---|---|---|---|---|
| Sample | at 40° C. | | at 40° C. | |
| name | T0 | T2W | T0 | T1M |
| P1 | 6.0 | 5.7 | 254 | 265 |
| P2 | 6.0 | 5.8 | 291 | 304 |
| P3 | 6.0 | 5.9 | 261 | 266 |
| P4 | 6.0 | 5.9 | 326 | 337 |
| P5 | 5.9 | 5.8 | 320 | 337 |
| P6 | 5.9 | 5.8 | 409 | 429 |
| P7 | 5.9 | 5.7 | 608 | 633 |
| P8 | 5.9 | 5.8 | 461 | 485 |
| P9 | 6.0 | 5.8 | 357 | 371 |
| P10 | 5.8 | 5.6 | 281 | 286 |
| P11 | 5.8 | 5.6 | 267 | 271 |
| P12 | 6.5 | 5.6 | 306 | 315 |
| P13 | 5.8 | 5.6 | 305 | 317 |
| P14 | 5.7 | 5.5 | 396 | 414 |
| P15 | 5.8 | 5.6 | 641 | 663 |
| P16 | 5.8 | 5.6 | 402 | 418 |

T0-represents data at zero time point;
M-indicates months

TABLE 25
SEC data of pembrolizumab formulations prepared as
per Example-6, when stored at 40° C. for one month.

| Sample | % HMW content | | % Monomer content | | % LMW content | |
|---|---|---|---|---|---|---|
| name | T0 | T1 M | T0 | T1 M | T0 | T1 M |
| P1 | 0.5 | 0.7 | 99.4 | 99.1 | 0.1 | 0.1 |
| P2 | 0.6 | 0.8 | 99.4 | 99.1 | 0.0 | 0.1 |
| P3 | 0.6 | 1.0 | 99.4 | 99.1 | 0.0 | 0.1 |
| P4 | 0.6 | 0.8 | 99.4 | 98.9 | 0.0 | 0.1 |
| P5 | 0.6 | 0.6 | 99.4 | 99.1 | 0.0 | 0.1 |
| P6 | 0.6 | 1.3 | 99.4 | 99.3 | 0.0 | 0.1 |
| P7 | 0.6 | 0.9 | 99.4 | 98.7 | 0.0 | 0.1 |
| P8 | 0.6 | 0.8 | 99.4 | 99.0 | 0.0 | 0.1 |
| P9 | 0.6 | 0.8 | 99.4 | 99.2 | 0.0 | 0.1 |
| P10 | 0.6 | 1.0 | 99.4 | 99.1 | 0.0 | 0.1 |
| P11 | 0.6 | 0.9 | 99.4 | 98.9 | 0.0 | 0.2 |
| P12 | 0.6 | 0.8 | 99.4 | 99.0 | 0.0 | 0.1 |
| P13 | 0.6 | 0.6 | 99.4 | 99.1 | 0.0 | 0.1 |
| P14 | 0.6 | 1.1 | 99.4 | 99.3 | 0.0 | 0.1 |
| P15 | 0.6 | 0.9 | 99.4 | 98.8 | 0.0 | 0.1 |
| P16 | 0.6 | 0.8 | 99.3 | 99.1 | 0.0 | 0.1 |

T0-represents data at zero time point;
M-indicates months

TABLE 26
IEX data of pembrolizumab formulations prepared as
per Example-6, when stored at 40° C. for one month.

| | % Acidic | | % Main peak | | % Basic variants | |
|---|---|---|---|---|---|---|
| Sample | variants | | content | | content | |
| name | T0 | T1 M | T0 | T1 M | T0 | T1 M |
| P1 | 11.7 | 22.0 | 67.0 | 60.1 | 21.4 | 28.2 |
| P2 | 10.2 | 18.5 | 66.6 | 61.3 | 23.2 | 31.5 |
| P3 | 10.5 | 19.9 | 66.5 | 61.3 | 23.0 | 31.1 |
| P4 | 10.8 | 19.6 | 67.1 | 61.3 | 22.1 | 30.5 |
| P5 | 11.0 | 20.2 | 67.3 | 61.8 | 21.7 | 30.2 |
| P6 | 10.9 | 19.1 | 67.0 | 61.2 | 22.1 | 30.6 |
| P7 | 10.9 | 18.5 | 66.7 | 60.8 | 22.4 | 30.9 |
| P8 | 10.9 | 22.9 | 66.2 | 61.6 | 23.0 | 29.0 |
| P9 | 11.0 | 20.8 | 66.8 | 62.8 | 22.3 | 29.1 |
| P10 | 11.1 | 21.9 | 66.6 | 61 | 22.2 | 31.1 |
| P11 | 11.0 | 27.5 | 67.1 | 60.8 | 21.9 | 28.7 |
| P12 | 11.2 | 21.8 | 67.2 | 61.4 | 21.6 | 25.7 |
| P13 | 11.2 | 23.0 | 66.8 | 61.1 | 22.0 | 28.1 |
| P14 | 11.3 | 19.9 | 66.9 | 60.8 | 21.7 | 26.0 |
| P15 | 11.5 | 24.1 | 67.1 | 59.7 | 21.4 | 29.4 |
| P16 | 11.6 | 21.9 | 66.6 | 60.7 | 23.2 | 27.8 |

T0-represents data at zero time point;
M-indicates months

TABLE 27
Opalescence of pembrolizumab samples prepared as per example-6.

| Sample | Opalescence at 40° C. | | | |
|---|---|---|---|---|
| Name | 0 W | 1W | 2W | 4W |

| | | |
|---|---|---|
| P1 | ROS-II-III and no visible | ROS-III-IV and no visible |
| | particles | particles |
| P2 | ROS-II-III and no visible | ROS-III-IV and no visible |
| | particles | particles |
| P3 | ROS-II-III and no visible | ROS-III-IV and small fibrous |
| | particles | particles |
| P4 | ROS-II-III and no visible | ROS-III-IV and small fibrous |
| | particles | particles |
| P5 | ROS-II-III and no visible | ROS-III-IV and no visible |
| | particles | particles |
| P6 | ROS-II-III and no visible | ROS-III-IV and small fibrous |
| | particles | particles |
| P7 | ROS-II-III and no visible | ROS-III-IV and small fibrous |
| | particles | particles |
| P8 | ROS-II-III and no visible | ROS-III-IV and no visible |
| | particles | particles |
| P9 | ROS-II-III and no visible | ROS-III-IV and no visible |
| | particles | particles |
| P10 | ROS-II-III and no visible | ROS-III-IV and small fibrous |
| | particles | particles observed |
| P11 | ROS-II-III and no visible | ROS-IV and no visible |
| | particles | particles |
| P12 | ROS-II-III and no visible | ROS-III-IV and small fibrous |
| | particles | particles |
| P13 | ROS-II-III and no visible | ROS-III-IV and small fibrous |
| | particles | particles |
| P14 | ROS-II-III and no visible | ROS-III-IV and no visible |
| | particles | particles |
| P15 | ROS-II-III and no visible | ROS-III-IV and no visible |
| | particles | particles |
| P16 | ROS-II-III and no visible | ROS-III-IV and no visible |
| | particles | particles |

### Example 7: High Concentration Pembrolizumab Formulations

Pembrolizumab in acetate buffer at a concentration of 35 mg/ml was buffer exchanged with succinate buffer followed by concentrating upto 250 mg/ml using centrifugation filters/ultrafiltration. Post which, concentration of the antibody was adjusted to 142 mg/ml using formulation buffer and various excipients such as sugar, amino acids and surfactant were added to prepare high concentration pembrolizumab formulation. Further, the formulation is subjected for accelerated stability conditions at 40° C. for one week. Details of the formulation along with quality attributes are given in below Table 27.

TABLE 27
Composition of high concentration pembrolizumab formulation prepared
as per Example-6, and quality attributes of the formulations

| | | | | | | | % Acidic | | % Main peak | | % Basic | |
|---|---|---|---|---|---|---|---|---|---|---|---|---|
| | pH at | | % HMW | | % Monomer | | variant | | content | | variants | |
| Sample | 40° C. | | at 40° C. | | at 40° C. | | at 40° C. | | at 40° C. | | at 40° C. | |
| name | T0 | T1 W | T0 | T1 W | T0 | T1 W | T0 | T1 W | T0 | T1 W | T0 | T1 W |
| 142 mg/ml | 5.61 | 5.52 | 0.8 | 0.9 | 99.2 | 99.1 | 6.2 | 8.0 | 65 | 65.4 | 28.8 | 28.6 |
| pembrolizumab, | | | | | | | | | | | | |
| 10 mM | | | | | | | | | | | | |
| succinate | | | | | | | | | | | | |
| buffer, 4.5% | | | | | | | | | | | | |
| trehalose, 0.8% | | | | | | | | | | | | |
| glycine and | | | | | | | | | | | | |
| 0.02% | | | | | | | | | | | | |
| polysorbate-80 | | | | | | | | | | | | |

1. A liquid pharmaceutical formulation of an anti-PDI antibody comprising, an anti-PDI antibody, succinate or acetate or citrate buffer having a pH of 5.0 to 6.0, sugar, amino acid, chelating agent and surfactant.

2. The formulation as claimed in claim 1, wherein the anti-PDI antibody concentration ranges from 10 mg/ml to 200 mg/ml.

3. A method of controlling sub-visible particle formation in an IgG4 anti-PDI antibody, the method comprising, formulating the IgG4 anti-PDI antibody in a composition comprising succinate or acetate or citrate buffer, sugar, chelating agent and surfactant.

4. The method as claimed in claim 3, wherein the composition further comprises methionine.

5. The method as claimed in claim 3, wherein the sub-visible particle size is >5 pm, or >10 pm, or >25 pm, or >50 pm and less than 80 pm.

6. The formulation or method as claimed in claim 1, wherein the anti-PDI antibody is nivolumab or pembrolizumab.

7. The formulation or method as claimed in claim 1, wherein the sugar is trehalose or sucrose.

8. The formulation or method as claimed in claim 1, wherein the chelating agent is ethylenediamine tetraacetic acid (EDTA) or diethylenetriamine pentaacetate (DTPA).

9. The formulation or method as claimed in claim 1, wherein the surfactant is polysorbate 80 or polysorbate 20.

10. A liquid pharmaceutical formulation of nivolumab antibody comprising nivolumab, succinate or acetate buffer, trehalose, methionine, sodium chloride, DTPA and surfactant, wherein the antibody concentration is in a range of 10 mg/ml to 200 mg/ml.

11. The formulation or method as claimed in claim 3, wherein the anti-PDI antibody is nivolumab or pembrolizumab.

12. The formulation or method as claimed in claim 3, wherein the sugar is trehalose or sucrose.

13. The formulation or method as claimed in claim 3, wherein the chelating agent is ethylenediamine tetraacetic acid (EDTA) or diethylenetriamine pentaacetate (DTPA).

14. The formulation or method as claimed in claim 3, wherein the surfactant is polysorbate 80 or polysorbate 20.
